(19) United States
(12) Patent Application Publication
(10) Pub. No.: US 2006/0211887 A1
(43) Pub. Date: Sep. 21, 2006

(54) Phosphate salts of 6-dimethylaminomethyl-1-(3-methoxyphenyl)-1,3-dihydroxy-cyclohexane compounds

(75) Inventors: Michael Gruss, Aachen (DE); Andreas Fischer, Huertgenwald (DE); Wolfgang Hell, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GMBH, AACHEN (DE)

(21) Appl. No.: 11/361,954

(22) Filed: Feb. 27, 2006

Related U.S. Application Data
(60) Provisional application No. 60/677,325, filed on May 4, 2005.

(30) Foreign Application Priority Data
Feb. 25, 2005 (DE) 10 2005 009 217.9

(51) Int. Cl. C07C 215/70

(52) U.S. Cl. 564/338

(57) ABSTRACT

Novel 6-dimethylaminomethyl-1-(3-methoxyphenyl)-1,3-dihydroxy-cyclohexane compounds in the form of phosphate salts, related polymorphs of these compounds, processes for their preparation, pharmaceutical formulations including these compounds and polymorphs and related methods of treating or inhibiting certain diseases or conditions.

### CROSS-REFERENCE TO RELATED APPLICATIONS

This application is an application claiming the benefit under 35 U.S.C. 119(e) of U.S. Provisional Application No. 60/677,325 filed May 4, 2005. This application also claims priority to German Patent Application No. 10 2005 009 217.9 filed Feb. 25, 2005.

### FIELD OF THE INVENTION

This invention relates to 6-dimethylaminomethyl-1-(3-methoxyphenyl)-1,3-dihydroxy-cyclohexane compounds (I) in the form of phosphate salts, processes for the preparation thereof and the use of these compounds in pharmaceutical formulations.

### BACKGROUND OF THE INVENTION

The treatment of chronic and non-chronic states of pain is of great importance in medicine. There is currently a worldwide need for additional pain therapy which is not exclusively opioid, but has a good action. The urgent need for a patient-oriented and targeted treatment of chronic and non-chronic states of pain, by which is to be understood successful and satisfactory pain treatment for patients, is documented in the large number of scientific works which have been published recently in the field of applied analgesics and of basic research into nociception.

Opioids have been employed for many years for pain treatment, although they cause a series of side effects, for example dependency, respiratory depression, a gastrointestinal inhibitory action and constipation. They can therefore be administered over a relatively long period of time or in relatively high dosages only under particular safety precautions, for example specific prescription instructions (Goodman, Gilman “The Pharmacological Basis of Therapeutics”, Pergamon Press, New York, 1990).

Tramadol hydrochloride—(1RS,2RS)-2[(dimethylamino)methyl]-1-(3-methoxyphenyl)-cyclohexanol, hydrochloride—occupies a special position among centrally acting analgesics, since this active compound causes a potent inhibition of pain without the side effects known for opioids (J. Pharmacol. Exp. Ther. 267, 331 (1993)). Tramadol is a racemate and consists of equal amounts of the (+)- and (−)-enantiomer. In vivo, the active compound forms the metabolite O-desmethyl-tramadol, which likewise is in the form of an enantiomer mixture. Investigations have shown that the enantiomers of tramadol as well as the enantiomers of the tramadol metabolites participate in the analgesic action (J. Pharmacol. Exp. Ther. 260, 275 (1992)).

In EP-B 0753506, substances having an analgesic action which are suitable for treatment of severe pain, without causing the side effects typical of opioids, were found.

EP-B 0753506 provides 6-dimethylaminomethyl-1-phenyl-cyclohexane compounds of the formula X 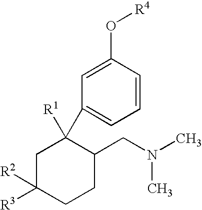
in which
R¹ is H, OH, Cl or F,
R² and R³ are identical or different and denote H, C₁₋₄-alkyl, benzyl, CF₃, OH, OCH₂—C₆H₅, OC₁₋₄-alkyl, Cl or F, with the proviso that at least one of the radicals R² or R³ denotes H,
R⁴ denotes H, CH₃, PO(OC₁₋₄-alkyl)₂, CO(OC₁₋₅-alkyl), CO—NH—C₆H₄—C₁₋₃-alkyl, CO—C₆H₄—R⁵, CO—C₁₋₅-alkyl, CO—CHR⁶—NHR⁷ or an unsubstituted or substituted pyridyl, thienyl, thiazoyl or phenyl group,
R⁵ denotes OC(O)C₁₋₃-alkyl in the ortho position or CH₂—N(R⁸)₂ in the meta or para position, wherein R⁸ represents C₁₋₄-alkyl or the two radicals R⁸, together with N, represent the 4-morpholino radical, and
R⁶ and R⁷ are identical or different and denote H or C₁₋₆-alkyl,
with the proviso that if the two radicals R² and R³ denote H, R⁴ is not CH₃ if R¹ denotes H, OH or Cl, or R⁴ is not H if R¹ denotes OH,
in the form of their bases or salts of physiologically acceptable acids.

The invention of the patent EP-B 0753506 furthermore provides a process for the preparation of 6-dimethylaminomethyl-1-phenyl-cyclohexane compounds of the formula X, in which R¹ denotes OH and R² and R³ are identical or different and denote H, C₁₋₄-alkyl, benzyl, CF₃, Cl or F, with the proviso that at least one of the radicals R² or R³ is H, and R⁴ denotes H, CH₃ or an unsubstituted or substituted pyridyl, thienyl, thiazoyl or phenyl group, with the proviso that R⁴ is neither CH₃ nor H if the two radicals R² and R³ denote H, the process comprising reacting a β-dimethylamino ketone of the formula II 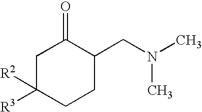
with an organometallic compound of the formula III 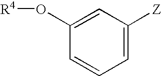
in which Z denotes MgCl, MgBr, MgI or Li, to give a compound of the formula X.

The invention of patent EP-B 0753506 additionally provides a process for the preparation of 6-dimethylaminomethyl-1-phenyl-cyclohexane compounds of the formula X in which R¹ is OH, one of the radicals R² or R³ denotes H and the other denotes OH, O—C₁₋₄-alkyl or OCH₂C₆H₅ and R⁴ denotes H, CH₃ or an unsubstituted or substituted pyridyl, thienyl, thiazoyl or phenyl group, wherein a β-dimethylaminoketone with a spirocyclic acetal structure of the formula V 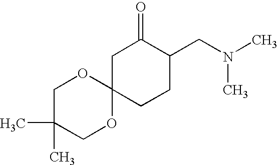
is reacted with an organometallic compound of the formula III 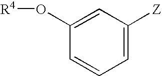
in which Z denotes MgCl, MgBr, MgI or Li, to form a compound of the formula VI, 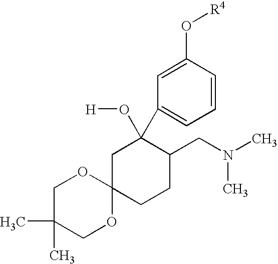
the obtained compound of the formula VI is converted by proton-catalyzed deacetalisation into the corresponding ketone derivative of the formula VIII 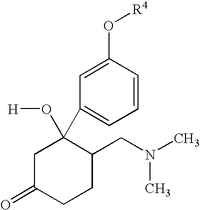
and the obtained ketone derivative is then reduced with a complex alkali metal hydride to form a compound of the formula I in which one of the radicals R² or R³ denotes OH, and optionally the compound of the formula I obtained by reduction is converted, after conversion into an alkali salt with a C₁₋₄-alkyl halide or benzyl halide, into a compound of the formula I in which one of the radicals R² or R³ denotes O—C₁₋₄-alkyl or OCH₂C₆H₅.

The compounds of EP-B 0753506 have a pronounced analgesic action and are toxicologically acceptable. They are therefore suitable as pharmaceutical active compounds. The invention accordingly also provides the use of a 6-dimethylaminomethyl-1-phenyl-1-cyclohexane compound of the formula X as an active compound in pharmaceutical formulations, preferably as an active compound in painkillers.

In EP-B 0753506, the compounds were converted with physiologically acceptable acids into their salts, the description listing: hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid and/or aspartic acid.

All the compounds in the embodiment examples in EP-B 0753506, e.g. Examples 18 and 19, are disclosed in the form of the HCl adduct, that is to say in the form of the salt adduct from the reaction of the compounds claimed in EP-B 0753506 with hydrochloric acid.

In the case of the 6-dimethylaminomethyl-1-(3-methoxyphenyl)-1,3-dihydroxy-cyclohexane compounds having a good action (Examples 18 and 19 from EP 0753506 B1), however, a large number of polymorphs and solvates (pseudopolymorphs) which can convert into one another are formed in the reaction to form the HCl adduct according to the synthesis instructions (see the parallel Application having the internal reference GRA 3110; application number EP 05004183.9 filed at the European Patent Office, Munich on 25.02.2005). This can represent a serious disadvantage, in particular in the use as a pharmaceutical formulation, since due to this polymorphism and pseudopolymorphism, certain polymorphic and solvated (pseudopolymorphic) forms of the HCl adduct can be prepared reproducibly only with difficulty by the preparation process disclosed in EP-B 0753506. A further property of the HCl salts of the compounds of Examples 18 and 19 from EP0753506 (6-dimethylaminomethyl-1-(3-methoxyphenyl)-1,3-dihydroxy-cyclohexane) is the marked tendency of these salts and their solvates to take up and release water, which can lead to problems during preparation and storage.

Related polymorphs of these compounds, processes for their preparation, pharmaceutical formulations including these compounds and polymorphs and related methods of treating or inhibiting certain diseases or conditions are also provided.

### SUMMARY OF THE INVENTION

One object of the present invention is to provide a form of the compound 6-dimethylaminomethyl-1-(3-methoxyphenyl) -1,3-dihydroxy-cyclohexane, which compound has a good action, which is physiologically acceptable and does not have the abovementioned disadvantages, namely which crystallizes in a dominant, polymorphic form and, in an optimum manner, shows a low hygroscopy and low tendency towards release of water under moderate environmental conditions, and therefore can also be readily and reproducibly prepared and stored without major changes.

This may be achieved, surprisingly, by providing the phosphate salt, which is not disclosed in EP-B 0753506, i.e. the reaction product of the 6-dimethylaminomethyl-1-(3-methoxyphenyl)-1,3-dihydroxy-cyclohexane compounds I with phosphoric acids to give the corresponding phosphate adduct.

The present Application therefore provides 6-dimethylaminomethyl-1-(3-methoxyphenyl) -1,3-dihydroxy-cyclohexane compounds of the formula I 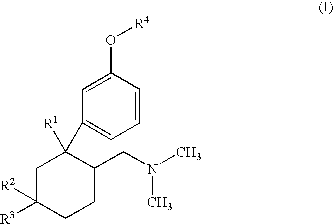
in which
R¹ denotes OH and
R² denotes OH and R³ denotes H or
R³ denotes OH and R² denotes H and
R⁴ denotes CH₃
   in the form of their phosphoric acid salts. In the following, the phosphate salts defined in this way are called phosphate salts I-P according to the invention.

### BRIEF DESCRIPTION OF THE DRAWINGS

FIG. 1 Powder diffractogram form A

FIG. 2 Powder diffractogram form B

FIG. 3 Powder diffractogram form C

FIG. 4 Powder diffractograms of the amorphous forms

FIG. 5 Raman spectrum P3

FIG. 6 Powder diffractogram P3

FIG. 7 Infrared spectra form A and form B (range 4000-1800 cm⁻¹)

FIG. 8 Infrared spectra form A and form B (range 1800-400 cm⁻¹)

FIG. 9 Raman spectra form A and form B (range 3500-400 cm⁻¹)

FIG. 10 Raman spectra form A and form B (range 3150-2750 cm⁻¹)

Table 1 Peak list powder diffractogram form A

Table 2 Peak list powder diffractogram form B

Table 3 Peak list powder diffractogram form C

Table 4 Peak list Raman spectrum P3

Table 5 Peak list powder diffractogram P3

### DETAILED DESCRIPTION

The person skilled in the art understands that the compounds of the above general formula (I) may, on account of their stereo centers, be present in each case in the form of one of their pure stereoisomers, in particular enantiomers or diastereomers, their racemates or in the form of a mixture of stereoisomers, in particular of the enantiomers and/or diastereomers, in an arbitrary mixture ratio in the salts according to the invention.

Phosphoric acids employed according to the invention are understood as meaning the oxo acids of phosphorus. The di- (also pyro-) and the condensed meta- and polyphosphoric acids, which are also included according to the invention, can be derived from orthophosphoric acid (relative molar mass 98.0 g/mole).

Primary, secondary and tertiary phosphates, which are also included according to the invention, can be formed by stepwise replacement of the H atoms of orthophosphoric acid.

Phosphate salts I-P according to the invention are understood as meaning salts from the reaction of I in particular with condensed phosphoric acids, such as meta- and diphosphoric acid, as well as salts of orthophosphoric acid.

Salts of diphosphoric acid and orthophosphoric acid are preferred.

Salts of orthophosphoric acid are most particularly preferred.

The present invention also provides phosphate salts I-P according to the invention, wherein the compounds in the salts have the configuration of the formula Ia 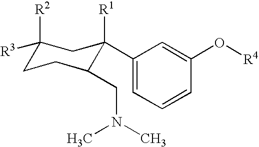
wherein preferably R¹ and R² in each case denote OH, R³ denotes H and R⁴ denotes CH₃.

Phosphate salts of the compounds (1RS,3RS,6RS)-6-dimethylaminomethyl-1-(3-methoxy-phenyl)cyclohexane-1,3-diol are preferred.

The orthophosphate salt of the racemic compound (1RS,3RS,6RS)-6-dimethylaminomethyl-1-(3-methoxyphenyl)-cyclohexane-1,3-diol of the following structure is particularly preferred: 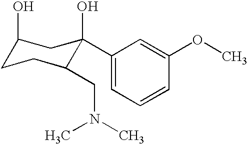
or, written another way:

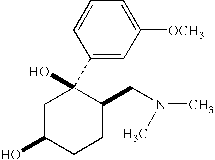

In a further embodiment the phosphates according to the invention, in particular orthophosphates, may comprise one of the enantiomers (+)-(1R,3R,6R)-6-dimethylaminomethyl-1-(3-methoxyphenyl)-cyclohexane-1,3-diol and (−)-(1S,3S,6S)-6-dimethylaminomethyl-1-(3-methoxyphenyl)-cyclohexane-1,3-diol or both of these enantiomers in a non-racemic mixture ratio.

The present invention additionally provides processes for the preparation of the phosphate salts I-P according to the invention, in which the reaction of a compound of the general formula (I) preferably takes place in a suitable reaction medium (conversion medium), preferably with phosphoric acid.

The present invention additionally provides a process for the preparation of a phosphate salt according to the invention, wherein another salt (i.e. different from the phosphate) of a compound of the general formula (I), in particular a salt of hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid and/or aspartic acid or the base, particularly preferably the hydrochloride or the free base I, is reacted with phosphoric acid, preferably in a molar ratio of I to phosphoric acid of 2:1 to 1:2, particularly preferably 1:1.5, and most particularly preferably 1.1:1 to 1:1.1.

In this connection the respective compound of the general formula (I) may be released in the form of the free base from the salt that is used, advantageously beforehand, in a conventional manner known to the person skilled in the art.

The present invention also provides a process for the preparation of the phosphate salts of I according to the invention, wherein the base I is suspended in alcohol, preferably isopropanol or ethanol, very preferably ethanol, at 10-40° C., preferably 20-30° C., very preferably 25° C., and dilute phosphoric acid is added, and the mixture is stirred at 0-10° C., preferably 5-7° C., and optionally seeded with the phosphate salt of I at 0-10° C., preferably 5-7° C. The product can then be filtered off with suction after 2-5 h, preferably 3-4 h, and dried.

A process for the preparation of the phosphate salts of I according to the invention may preferably be used, in which dilute phosphoric acid is added to the base I at 20-30° C. in isopropanol and/or ethanol, optionally mixed with water, and the mixture is stirred at 0-10° C. and is optionally seeded with the phosphate salt of I at 0-10° C. The product can then be filtered off under suction after 2-5 hours and dried.

The present invention also provides a pharmaceutical formulation comprising at least one phosphate salt I-P according to the invention, in each case optionally in the form of one of its pure stereoisomers, in particular enantiomers or diastereomers, its racemates or in the form of a mixture of stereoisomers, in particular the enantiomers and/or diastereomers, in any desired mixture ratio, or in each case in the form of a corresponding solvate, and optionally one or more pharmaceutically acceptable auxiliary substances.

The pharmaceutical formulation according to the invention is preferably suitable for the prophylaxis and/or treatment of pain, preferably chosen from the group consisting of acute pain, chronic pain, neuropathic pain and visceral pain; of migraine; depressions; neurodegenerative diseases, preferably chosen from the group consisting of Parkinson's disease, Alzheimer's disease, Huntington's disease and multiple sclerosis; cognitive diseases, preferably cognitive deficiency states, particularly preferably attention deficit syndrome (ADS); panic attacks; epilepsy; coughing; urinary incontinence; diarrhea; pruritus; schizophrenia; cerebral ischaemias; muscle spasms; spasms; eating disorders, preferably chosen from the group consisting of bulimia, cachexia, anorexia and obesity; alcohol and/or drug (in particular nicotine and/or cocaine) and/or pharmaceutical formulation abuse; alcohol and/or drug (in particular nicotine and/or cocaine) and/or pharmaceutical formulation dependency, preferably for the prophylaxis and/or reduction of withdrawal symptoms of alcohol and/or drug (in particular nicotine and/or cocaine) and/or pharmaceutical formulation dependency; development of tolerance symptoms to pharmaceutical formulations; in particular to opioids; gastro-esophageal reflux syndrome; for diuresis; for antinatriuresis; for influencing the cardiovascular system; for anxiolysis; for increasing vigilance; for increasing libido, for modulation of motor activity and for local anesthesia.

The pharmaceutical formulation according to the invention is particularly preferably suitable for the prophylaxis and/or treatment of pain, preferably acute pain, chronic pain, neuropathic pain or visceral pain; depressions; epilepsy; Parkinson's disease; alcohol and/or drug (in particular nicotine and/or cocaine) and/or pharmaceutical formulation abuse; alcohol and/or drug (in particular nicotine and/or cocaine) and/or pharmaceutical formulation dependency; preferably for the prophylaxis and/or reduction of withdrawal symptoms with alcohol and/or drug (in particular nicotine and/or cocaine) and/or pharmaceutical formulation dependency; development of tolerance symptoms to pharmaceutical formulations, in particular to opioids, or for anxiolysis.

The pharmaceutical formulation according to the invention is very particularly preferably suitable for the prophylaxis and/or treatment of pain, preferably acute pain, chronic pain, neuropathic pain or visceral pain.

The pharmaceutical formulation according to the invention is most particularly preferably suitable also for the prophylaxis and/or treatment of pain due to inflammation.

Particularly preferred is the use of at least one phosphate salt according to the invention, in each case optionally in the form of one of its pure stereoisomers, in particular enantiomers or diastereomers, its racemates or in the form of a mixture of stereoisomers, in particular the enantiomers and/or diastereomers, in any desired mixture ratio, or in each case in the form of a corresponding solvate, and optionally one or more pharmaceutically acceptable auxiliary substances, for the preparation of a pharmaceutical formulation for the prophylaxis and/or treatment of pain, preferably chosen from the group consisting of acute pain, chronic pain, neuropathic pain and visceral pain, of migraine, depressions, neurodegenerative diseases, preferably chosen from the group consisting of Parkinson's disease, Alzheimer's disease, Huntington's disease and multiple sclerosis, cognitive diseases, preferably cognitive deficiency states, particularly preferably attention deficit syndrome (ADS), panic attacks, epilepsy, coughing, urinary incontinence, diarrhea, pruritus, schizophrenia, cerebral ischaemias, muscle spasms, spasms, eating disorders, preferably chosen from the group consisting of bulimia, cachexia, anorexia and obesity, alcohol and/or drug (in particular nicotine and/or cocaine) and/or pharmaceutical formulation abuse, alcohol and/or drug (in particular for the prophylaxis and/or reduction of withdrawal symptoms in alcohol and/or drug (in particular nicotine and/or cocaine) and/or pharmaceutical formulation dependency, development of tolerance symptoms to drugs and/or pharmaceutical formulations, in particular to opioids, gastro-esophageal reflux syndrome, for diuresis, for antinatriuresis, for influencing the cardiovascular system, for anxiolysis, for increasing vigilance, for increasing libido, for modulation of motor activity and for local anesthesia.

The pharmaceutical formulation according to the invention can be in a liquid, semi-solid or solid pharmaceutical formulation form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, optionally pressed to tablets, filled in capsules or suspended in a liquid, and can also be administered as such.

In addition to at least one phosphate salt according to the invention, optionally in the form of its pure stereoisomers, in particular enantiomers or diastereomers, its racemates or in the form of mixtures of the stereoisomers, in particular the enantiomers or diastereomers, in any desired mixture ratio, or in each case in the form of a corresponding solvate, the pharmaceutical formulation according to the invention conventionally comprises further physiologically acceptable pharmaceutical auxiliary substances, which can preferably be chosen from the group consisting of carrier materials, fillers, solvents, diluents, surface-active substances, dyestuffs, preservatives, disintegrating agents, slip agents, lubricants, aroma substances and binders.

The choice of the physiologically acceptable auxiliary substances and the amounts thereof to be employed depends on whether the pharmaceutical formulation is to be administered orally, subcutaneously, parenterally, intravenously, intraperitoneally, intradermally, intramuscularly, intranasally, buccally, rectally or locally, for example on infections on the skin, the mucous membranes and on the eyes. Formulations in the form of tablets, coated tablets, capsules, granules, pellets, drops, juices and syrups are preferably suitable for oral administration, and solutions, suspensions, easily reconstitutable dry formulations and sprays are suitable for parenteral, topical and inhalatory administration.

Depôt formulations in dissolved form or in a plaster, optionally with the addition of agents which promote penetration through the skin, are also suitable formulations for percutaneous administration.

Formulation forms which can be used orally or percutaneously can release the particular phosphate salts according to the invention in a delayed manner.

The pharmaceutical formulations according to the invention are prepared by means of conventional means, devices, methods and processes which are well-known from the prior art, such as are described, for example, in “Remington's Pharmaceutical Sciences”, editor A. R. Gennaro, 17th edition, Mack Publishing Company, Easton, Pa., 1985, in particular in Part 8, Chapters 76 to 93. The corresponding description is introduced herewith as reference and forms part of the disclosure.

The amount of the particular phosphate salt according to the invention to be administered to patients can vary, and depends for example on the weight or age of the patient and on the mode of administration, the indication and the severity of the disease. 0.005 to 5,000 mg/kg, preferably 0.05 to 500 mg/kg, particularly preferably 0.1 to 50 mg/kg of patient's body weight of at least one such compound are conventionally administered.

Experimental evidence which demonstrates the advantages of the phosphate salt I-P according to the invention compared with the HCl salt disclosed in EP-B 0753506, identified I-H hereinafter, is provided in the following. The HCl salt I—H prepared according to EP-B 0753506 and the phosphate salt I-P prepared according to the invention are compared with one another.

The HCl salt I-H prepared according to EP-B 0753506 is first exposed to defined atmospheric humidities for certain periods of time and then dried (Table 1-4; for the synthesis of the compounds see the following experimental part; for the definition of the polymorphs see Application EP 05004183.9 filed on 25 Feb. 2006, internal reference GRA 3110).

### COMPARASION EXAMPLES I-H

Storage of the hydrochloride salts, which are not according to the invention (identified H1 to H3 in the following examples of compounds; for the synthesis see the subsequent experimental part), at room temperature over saturated sodium chloride solution results in a relative atmospheric humidity of approx. 75% (±5%). Storage of the samples at room temperature over saturated potassium sulfate solution results in a relative atmospheric humidity of approx. 95% (±5%).

Storage of the samples at room temperature over drying beads (blau+, Engelhardt, Nienburg) results in a relative atmospheric humidity of approx. 4% (±5%).

### Table 1

### COMPARISON EXAMPLES HCl SALT I-H

The samples were stored at approx. 75% relative atmospheric humidity for approx. 8.5 days. The samples were then stored at a relative atmospheric humidity of approx. 4% for a further approx. 6 h.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage to before the particular storage step is stated.

In addition, the percentage change in the total weight of the sample occurring in the respective storage step was determined by weighing (“laboratory”).

The crystalline form was determined by means of X-ray powder diffractometry.

TABLE 2
(Comparison Examples HCl salt I—H)

| | Form | | | |
|---|---|---|---|---|
| Sample | before | Form after | % pt | Laboratory/% |
| H1 | form A, | form B, further | −1.22 | +1.14 |
| | form D | peaks | | |
| H2 | form A, | form B, further | −2.85 | +2.61 |
| | form C, | peaks | | |
| | form D | | | |

The samples were stored at approx. 95% relative atmospheric humidity for approx. 8.5 days. The samples were then stored at a relative atmospheric humidity of approx. 4% for a further approx. 6 h.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage to before the storage is stated.

In addition, the percentage change in the total weight of the sample occurring in the respective storage step was determined by weighing (“laboratory”).

The crystalline form was determined by means of X-ray powder diffractometry.

| | Form | | | |
|---|---|---|---|---|
| Sample | before | Form after | % pt | Laboratory/% |
| H1 | form A, | form B, further | −4.89 | +4.99 |
| | form D | peaks | | |
| H2 | form A, | form B, further | −4.95 | +4.88 |
| | form C, | peaks | | |
| | form D | | | |

The samples were stored at approx. 75% relative atmospheric humidity for approx. 7 days. The samples were then stored at a relative atmospheric humidity of approx. 4% for a further approx. 2.5 days.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage step to before the storage is stated.

In addition, the percentage change in the total weight of the sample occurring in the respective storage step was determined by weighing (“laboratory”).

The crystalline form was determined by means of X-ray powder diffractometry.

TABLE 4
(Comparison Examples HCl salt I—H)

| Sam- | Form | Form | | Labo- | Form | | Labo- |
|---|---|---|---|---|---|---|---|
| ple | before | middle | % pt | ratory/% | after | % pt | ratory/% |
| H1 | form A, | form A, | −2.73 | 2.89% | form B, | 0.53 | −1.24 |
| | form D | form B, | | | form A | | |
| | | form C, | | | | | |
| | | form D | | | | | |
| H3 | form B | form B | −4.75 | +0.02 | form B | 0.46 | +3.68 |

### Table 4

### COMPARATIVE EXAMPLES HCl SALT I-H

The samples were stored at approx. 95% relative atmospheric humidity for approx. 7 days. The samples were then stored at a relative atmospheric humidity of approx. 4% for a further approx. 7 days.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage step to before the storage is stated.

In addition, the percentage change in the total weight of the sample occurring in the respective storage step was determined by weighing (“laboratory”).

The crystalline form was determined by means of X-ray powder diffractometry.

| Sam- | Form | Form | | Labo- | Form | | Labo- |
|---|---|---|---|---|---|---|---|
| ple | before | middle | % pt | ratory/% | after | % pt | ratory/% |
| H1 | form A, | form B | −4.88 | +13.72 | form B | −3.84 | −22.76 |
| | form D | | | | | | |
| H3 | form B | form B | −2.37 | +4.34 | form B | −0.49 | −9.19 |

It can be seen from Tables 1-4 that the HCl adduct (Comparison Examples I-H), which crystallizes in various polymorphic forms, initially takes up water, and this uptake occurs to a different degree, depending on the atmospheric humidity and the polymorph/polymorph mixture employed. Depending on the polymorphic form A, B, C or D employed, a uniform polymorph is no longer obtained at the end after the drying, but instead various polymorph mixtures, which also have various and in some cases non-reproducible water contents after drying, are obtained.

In contrast, in the case of the phosphate salts I-P according to the invention of the compound 6-dimethylaminomethyl-1-(3-methoxyphenyl)-1,3-dihydroxy-cyclohexane, both after exposure to atmospheric humidity of 75% and 95% for a defined period of time the form A is found in an identical manner, without water having been taken up, and after drying by exposure to a relative atmospheric humidity of approx. 4% at room temperature the same form A, which has a constant and reproducible water content, is present again. This is demonstrated by the following overview (Table 5-8; for the synthesis of samples P1, P2, P3 see the experimental part):

Storage of the phosphate salts I-P according to the invention, called samples in the following, at room temperature over saturated sodium chloride solution results in a relative atmospheric humidity of approx. 75 (±5) % . Storage of the samples at room temperature over saturated potassium sulfate solution results in a relative atmospheric humidity of approx. 95 (±5) %.

Storage of the samples at room temperature over drying beads (blau+, Engelhardt, Nienburg) results in a relative atmospheric humidity of approx. 4 (±5) %.

Table 5 (According to the Invention)

The samples were stored at approx. 75 (±5) % relative atmospheric humidity for approx. 8.5 days. The samples were then stored at a relative atmospheric humidity of approx. 4 (±5) % for a further approx. 6 h.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage to before the storage is stated.

In addition, the percentage change in the total weight of the sample occurring in the respective storage step was determined by weighing (“laboratory”).

The crystalline form was determined by means of X-ray powder diffractometry.

TABLE 6
(according to the invention)

| | Form | | | |
|---|---|---|---|---|
| Sample | before | Form after | % pt | Laboratory |
| P1 | form A | form A | −0.05 | −0.01% |
| P2 | form A | form A | −0.17 | +0.06% |
| P3 | form A | form A | −0.24 | −0.47% |

The samples were stored at approx. 95 (±5) % relative atmospheric humidity for approx. 8.5 days. The samples were then stored at a relative atmospheric humidity of approx. 4 (±5) % for a further approx. 6 h.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage to before the storage is stated.

In addition, the percentage change in the total weight of the sample occurring in the respective storage step was determined by weighing (“laboratory”).

The crystalline form was determined by means of X-ray powder diffractometry.

TABLE 7
(according to the invention)

| Sample | Form before | Form after | % pt | Laboratory |
|---|---|---|---|---|
| P1 | form A | form A | −0.22 | +0.12% |
| P2 | form A | form A | −0.25 | +0.02% |
| P3 | form A | form A | −0.25 | −0.39% |

The samples were stored at approx. 75 (±5) % relative atmospheric humidity for approx. 7 days. The samples were then stored at a relative atmospheric humidity of approx. 4 (±5) % for a further approx. 20 h.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage step to before the storage is stated.

In addition, the percentage change in the total weight of the sample occurring in the respective storage step was determined by weighing (“laboratory”).

The crystalline form was determined by means of X-ray powder diffractometry.

TABLE 8
(according to the invention)

| | | | | Labo- | | | Labo- |
|---|---|---|---|---|---|---|---|
| Sam- | Form | Form | % pt | ratory | Form | % pt | ratory |
| ple | before | during | during | during % | after | after | after |
| P1 | form A | form A | −0.21 | −0.08% | form A | −0.04 | +0.07% |
| P2 | form A | form A | −0.42 | +0.02% | form A | −0.44 | +0.08% |
| P3 | form A | form A | −0.25 | +0.23% | form A | −0.33 | −0.75% |

The samples were stored at approx. 95 (±5) % relative atmospheric humidity for approx. 7 days. The samples were then stored at a relative atmospheric humidity of approx. 4 (±5) % for a further approx. 20 h.

They were analyzed for their loss in weight by means of thermogravimetric analyses (amount of sample approx. 5-20 mg, heating rate approx. 10 K/min, heating range from approx. 25° C. to approx. 240° C.).

The approximate difference in the moisture content (in percentage points; % pt) from after the storage step to before the storage is stated.

In addition, the percentage change in the total weight of the sample occurring in the respective storage step was determined by weighing (“laboratory”).

The crystalline form was determined by means of X-ray powder diffractometry.

| | | | | Labo- | | | Labo- |
|---|---|---|---|---|---|---|---|
| Sam- | Form | Form | % pt | ratory | Form | % pt | ratory |
| ple | before | during | during | during | after | after | after |
| P1 | form A | form A | −0.19 | +2.16% | form A | −0.02 | −0.04% |
| P2 | form A | form A | −0.24 | +0.52% | form A | −0.21 | −0.60% |
| P3 | form A | form A | +0.21 | 5.33% | form A | +0.27 | −0.04% |

Sample P3 was kept in the laboratory for approx. 55 minutes under ambient conditions before the second storage step.

The interpretation of the specified data is explained again in more detail by the example of the data for P1 given in the preceding Table 8:

Storage of 1:
first storage step: 95% atmospheric humidity
second storage step: 4% atmospheric humidity

Sample: Identification of the sample
Form beforehand: polymorphic form of the material before storage,
determined by means of X-ray powder diffractometry.

Laboratory:
Weight of the sample before storage at 95%=119.58 mg
Weight of the sample after storage at 9%=122.16 mg
   =>weight change=((122.16 mg−119.58 mg)×100)/119.58 mg=+2.16%
   Material was then taken from the sample for instrumental analysis.

Intermediate form: polymorphic form of the material after the storage step at high atmospheric humidity determined by means of X-ray powder diffractometry
% Pt: difference of the weight losses determined by thermogravimetry.
Weight loss TG (beforehand)=−4.36%
Weight loss TG (intermediate)=−4.55%
   =>% Pt=(TG (intermediate)−TG (beforehand))×100−0.19% Pt=(−4.55%−−4.36%)×100

The second storage step is carried out at ca. 4% atmospheric humidity.

Laboratory:
Weight of the sample before storage at 4%=73.40 mg
Weight of the sample after storage at 4%=73.37 mg
   =>weight change=((73.37 mg−73.40 mg)×100)/73.40 mg=−0.04%
   Material from the sample was then taken for instrumental analysis.

Form afterwards: polymorphic form of the material after this storage step at low atmospheric humidity, determined by means of X-ray powder diffractometry.
% Pt: difference of the weight losses determined by thermogravimetry.
Weight loss TG (beforehand)=−4.36% (same value as above TG (beforehand))
Weight loss TG (afterwards)=−4.38%
   =>% Pt=(TG (afterwards)−TG (beforehand))×100−0.02% Pt=(−4.38%−−4.36% )×100

The other data given in Tables 1-8 are also appropriately specified in each case.

The comparison shows that, in contrast to the HCl adduct I-H, the phosphate salt I-P according to the invention can be employed and can be stored with a defined stoichiometry. Furthermore, the stable form of polymorph A, which can no longer be converted into other polymorphs in a wide range of ambient conditions (it is potentially possible to obtain, by specific conditions, the amorphous form or, in suspension in acetonitrile, another solvate) regularly preferably forms under conditions of the preparation process according to the invention, in contrast to the case, which is not according to the invention, of the HCl adducts of 6-dimethylaminomethyl-1-(3-methoxyphenyl)-1,3-dihydroxy-cyclohexane. The HCl salt mixtures prepared according to EP-B 0753506, which vary non-reproducibly, e.g. form A and C convert into form B, or form A, C and D convert into form B, or form A, C and D convert into form B and A or also only into form B, take up non-reproducible amounts of water under conditions with increased atmospheric humidities (from a lower limit of approx. 60% r.h. up to an upper limit of approx. 100% r.h., particularly in the range of approx. 70—approx. 100% r.h., very particularly in the range of approx. 75—approx. 100% r.h.).

Preparation of the “main polymorph A” and further polymorphs B, C and the “amorphous” form of the phosphate salts according to the invention:

By precipitation of the free base I with phosphoric acids according to the invention, preferably orthophosphoric acid, or reactions of the HCl adduct of I with phosphoric acids, the dominant polymorphic form A, the data of which are stated in Example 7, is regularly formed, preferably under conditions according to the invention, that is to say in a molar ratio of base I to phosphoric acid in the range of from 2:1 to 1:2, particularly preferably 1:1.5; very particularly preferably 1.1:1 to 1:1.1). In addition the robustness of the synthesis of polymorph A with respect to variations in the reaction conditions is shown in Examples 5, 6, 8 and 9.

By controlled specific manipulations of the reaction conditions, in deviation from the reaction conditions according to the invention, further, in some cases unstable polymorphs can also be produced: Examples 10, 11, 12 (form B; acetonitrile solvate), Example 16 (form C; metastable), Examples 15, 18, 19 (amorphous form). In Example 21, X-ray diffractograms of forms A, B, C and the amorphous form are shown for characterization, and in Examples 22 and 23 the result of a comparative IR and, respectively, RAMAN analysis is shown.

The preferred solvents for producing the I-P polymorphs A, B, C and the amorphous form are stated in the following:

Form A: Preparation from a solution or suspension of the base of I in organic solvents or water or mixtures thereof. The solvents may preferably be chosen from water; methanol; ethanol; 1-propanol; 2-propanol; acetone; ethyl acetate; hexane; 2-butanone; toluene; tetrahydrofuran; isopropyl ether; 1,4-dioxane; 1-propanol; 1-butanol; 2-methyl-1-propanol; 1-pentanol; 3-methyl-1-butanol; diethyl ether; (tert-butyl) methyl ether; tetrahydrofuran; methoxybenzene; 4-methyl-2-pentanone, iso-butyl methyl ketone; formic acid; acetic acid; ethyl formate; methyl acetate; ethyl acetate; n-propyl acetate; n-butyl acetate; methylene chloride; dimethyl sulfoxide; (E)-1,2-dichloroethene; (Z)-1,2-dichloroethene; trichloroethene; toluene; chlorobenzene; pyridine; 2-methoxyethanol; 1,2-ethanediol, glycol; 1,2-dimethoxyethane; 1,4-dioxane; 3,3-dimethyl-2-butanone, tert-butyl methyl ketone; formamide; N,N-dimethylformamide; N,N-dimethylacetamide; 1-methylpyrrolidin-2-one;

or mixtures thereof,

preferably:

water; methanol; ethanol; 1-propanol; 2-propanol; acetone; ethyl acetate; hexane; 2-butanone; toluene; tetrahydrofuran; isopropyl ether; 1,4-dioxane; 1-propanol; 1-butanol; 2-methyl-1-propanol; 1-pentanol; 3-methyl-1-butanol; diethyl ether; (tert-butyl) methyl ether; tetrahydrofuran; methoxybenzene; 4-methyl-2-pentanone, iso-butyl methyl ketone; formic acid; acetic acid; ethyl formate; methyl acetate; ethyl acetate; n-propyl acetate; n-butyl acetate; methylene chloride; dimethyl sulfoxide;

or mixtures thereof,

most preferably:

water; methanol; ethanol; 1-propanol; 2-propanol; acetone; ethyl acetate; hexane; 2-butanone or mixtures thereof.

Form B: Preferably from acetonitrile or mixtures of acetonitrile and organic solvents or water.

“Amorphous” polymorph:

preferably: water; methanol; ethanol; 1-propanol; 2-propanol; acetone; ethyl acetate; hexane; 2-butanone; toluene; tetrahydrofuran; isopropyl ether; 1,4-dioxane; 1-propanol; 1-butanol; 2-methyl-1-propanol; 1-pentanol; 3-methyl-1-butanol; diethyl ether; (tert-butyl) methyl ether; tetrahydrofuran; methoxybenzene; 4-methyl-2-pentanone; formic acid; acetic acid; ethyl formate; methyl acetate; ethyl acetate; n-propyl acetate; n-butyl acetate; methylene chloride; dimethyl sulfoxide or mixtures thereof, extremely preferably: acetonitrile, water; methanol; ethanol; 2-propanol or mixtures thereof.

The present Application furthermore provides all the polymorphs of I-P, in particular polymorph A, B, C, the “amorphous” form and mixtures thereof, polymorph A being particularly preferred.

The present invention furthermore provides processes for the preparation of the polymorphs of I-P.

The present invention furthermore provides pharmaceutical compositions comprising one or more polymorphs from the group A, B, C and the “amorphous” form, preferably A. The present invention furthermore provides the use of one or more polymorphs of I-P for the preparation of a pharmaceutical formulation for the treatment of pain, incontinence, depression and anxiety states, preferably pain, particularly preferably acute and chronic pain.

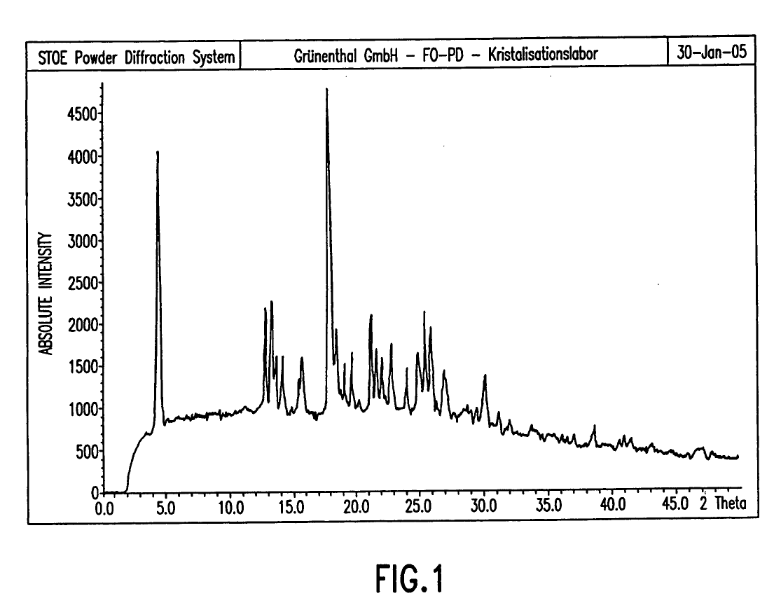

The present Application furthermore provides polymorph A of the orthophosphate I-P, which has a powder diagram as shown in FIG. 1, measured with Cu Kalpha radiation.

The present Application furthermore provides polymorph A of the orthophosphate I-P, which has peaks corresponding to Table 1 measured in the powder diffractogram, measured with Cu Kalpha radiation.

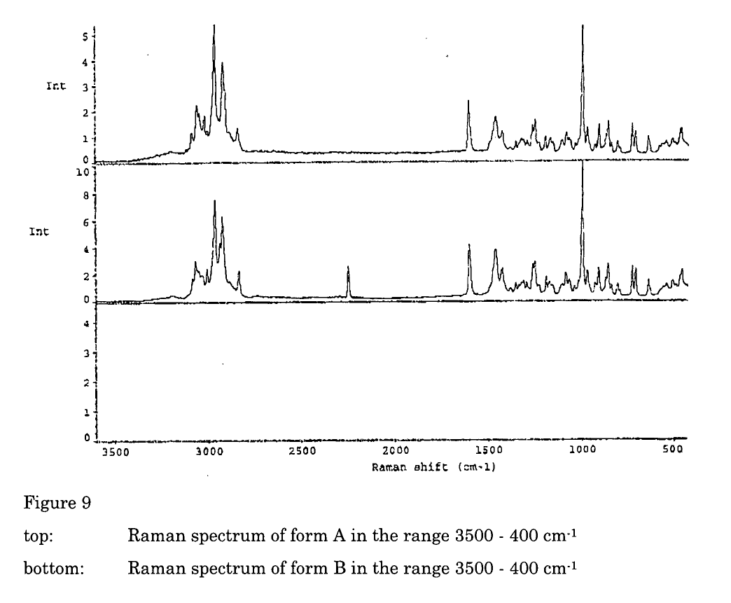

The present Application furthermore provides polymorph A of the orthophosphate I-P, which has a RAMAN spectrum measured at 1064 nm, as shown in FIG. 9.

The application furthermore provides polymorph A of the orthophosphate salt of (1RS,3RS,6RS)-6-dimethylaminomethyl-1-(3-methoxyphenyl)-cyclohexane-1,3-diol, which has a powder diffractogram comprising one or both of the following reflections: 30.0 and 33.7 (in each case ±0.2 2θ). The powder diffractogram may preferably contain in addition one or more of the following reflections: 4.6, 13.8, 15.6, 15.9, 18.0, 18.4, 19.1, 19.6, 21.6, 24.9 and 32.0 (in each case ±0.2 2θ).

The application furthermore provides polymorph A of the orthophosphate salt of (1RS,3RS,6RS)-6-dimethylaminomethyl-1-(3-methoxyphenyl)-cyclohexane-1,3-diol, which has a Raman spectrum containing one or more of the following signals: 2912, 3020 and 3060 (in each case in cm⁻¹±4 cm⁻¹). The Raman spectrum may preferably also include one or more of the following signals: 2843, 2922, 2966 and 3089 (in each case in cm⁻¹+−4 cm⁻¹).

The application furthermore provides a process for the preparation of polymorph A, according to which (1RS,3RS,6RS)-6-dimethylaminomethyl-1-(3-methoxyphenyl)-cyclohexane-1,3-diol is reacted with orthophosphoric acid in a reaction medium and the polymorph A that is thereby obtained is optionally purified and isolated.

In a preferred embodiment of the process (1RS,3RS,6RS)-6-dimethylaminomethyl-1-(3-methoxyphenyl)-cyclohexane-1,3-diol and orthophosphoric acid may be used in a molar ratio of 2:1 to 1:2, preferably 1.5:1 to 1:1.5, particularly preferably 1.1:1 to 1:1.1.

In a similarly preferred embodiment of the process the reaction may be carried out at a temperature of 10-40° C., preferably 20-30° C., most preferably at ca. 25° C.

In a likewise preferred embodiment of the process an alcohol may be used as reaction medium, optionally mixed with water, preferably isopropanol and/or ethanol optionally mixed with water, most particularly preferably ethanol optionally mixed with water.

In a likewise preferred embodiment of the process the mixture of (1RS, 3RS,6RS)-6-dimethylaminomethyl-1-(3-methoxyphenyl)-cyclohexane-1,3-diol and orthophosphoric acid may be stirred at 0-10° C., preferably 5-7° C., and optionally seeded with polymorph A at 0-10° C., preferably 5-7° C.

The application also provides polymorph A obtainable according to one of the processes described hereinbefore.

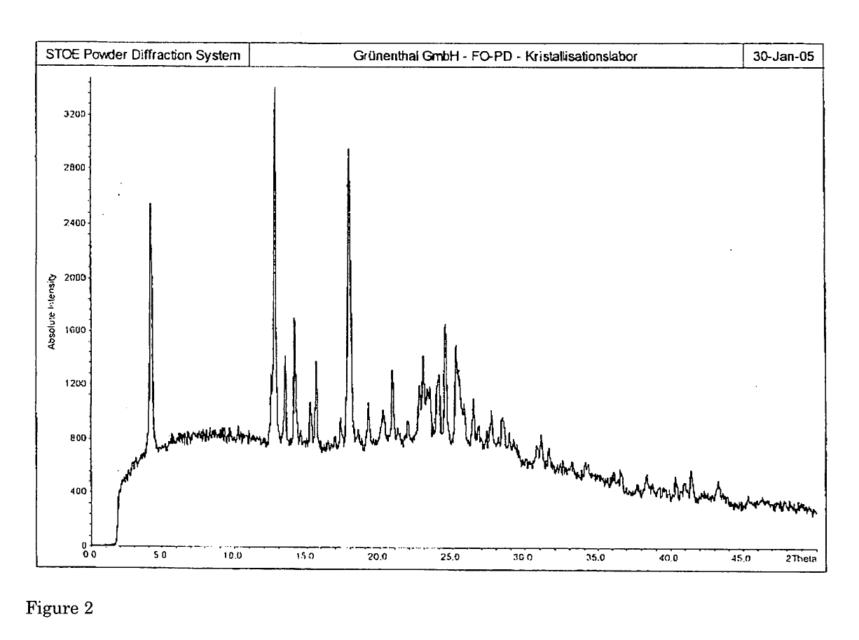

The present Application furthermore provides polymorph B of the orthophosphate I-P, which has a powder diagram as shown in FIG. 2, measured with Cu Kalpha radiation.

The present Application furthermore provides polymorph B of the orthophosphate I-P, which has peaks corresponding to Table 2 measured in the powder diffractogram, measured with Cu Kalpha radiation.

The present Application furthermore provides polymorph B of the orthophosphate I-P, which has a RAMAN spectrum measured at 1064 nm, as shown in FIG. 9.

The application furthermore provides polymorph B of the orthophosphate salt of (1RS,3RS,6RS)-6-dimethylaminomethyl-1-(3-methoxyphenyl)-cyclohexane-1,3-diol, characterized by a powder diffractogram containing one or more of the following reflections: 17.0, 17.4 and 20.2 (in each case ±0.2 to 2θ). The powder diffractogram may preferably contain in addition one or more of the following reflections: 4.3, 14.6, 15.2, 15.6, 18.0 and 31.6 (in each case ±0.2 to 2θ).

The application furthermore provides polymorph B of the orthophosphate salt of (1RS,3RS,6RS)-6-dimethylaminomethyl-1-(3-methoxyphenyl)-cyclohexane-1,3-diol, characterized by a Raman spectrum containing one or both of the following signals: 2940 and 3070 (in each case in cm⁻¹±4 cm⁻²). The Raman spectrum may preferably also include one or more of the following signals: 2839, 2926, 2964 and 3084 (in each case in cm⁻¹ ±4 cm⁻¹).

The application furthermore provides a process for the preparation of polymorph B, according to which polymorph A is stirred in acetonitrile or in a medium based on acetonitrile, optionally at elevated temperature, and the polymorph B that is thereby obtained is isolated.

In a preferred embodiment of the process the medium based on acetonitrile may contain >50 vol. % , preferably >75 vol. % , of acetonitrile.

In a likewise preferred embodiment of the process the medium may contain, apart from acetonitrile, also an alcohol, preferably ethanol.

In a similarly preferred embodiment of the process the reaction to form polymorph B may be carried out at a temperature from 10° to 60° C., preferably 20° to 50° C.

In a likewise preferred embodiment of the process polymorph B may, after isolation, be dried at a temperature of ≦60° C., preferably ≦40° C., optionally under reduced pressure.

The application furthermore provides polymorph B obtainable according to one of the aforedescribed processes.

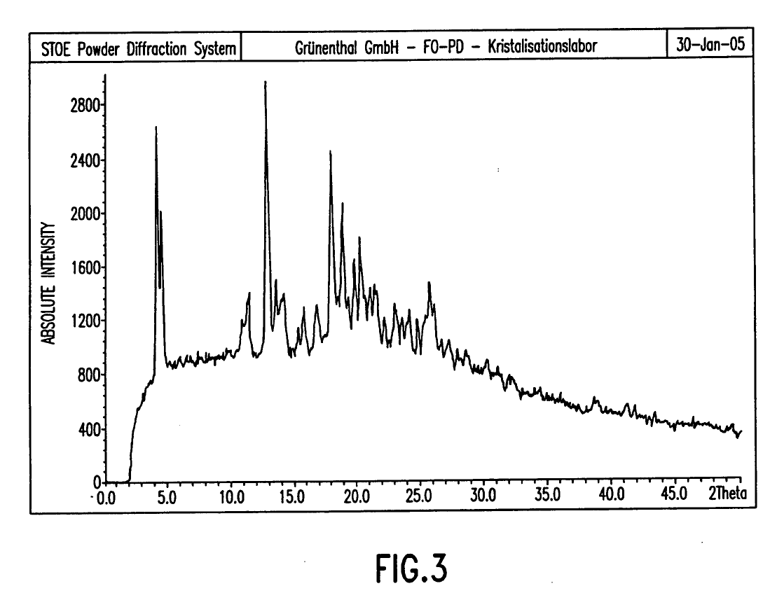

The present Application furthermore provides polymorph C of the orthophosphate I-P, which has a powder diagram as shown in FIG. 3, measured with Cu Kalpha radiation.

The present Application furthermore provides polymorph C of the orthophosphate I-P, which has peaks corresponding to Table 3 measured in the powder diffractogram, measured with Cu Kalpha radiation.

The application furthermore provides polymorph C of the orthophosphate salt of (1RS,3RS,6RS)-6-dimethylaminomethyl-1-(3-methoxyphenyl)-cyclohexane-1,3-diol, characterized by a powder diffractogram comprising one or both of the following reflections: 10.7 and 11.4 (in each case ±0.2 2θ). The powder diffractogram may preferably, contain in addition one or both of the following reflections: 16.7 and 18.8 (in each case ±0.2 2θ).

The application furthermore provides a process for the preparation of polymorph C, according to which less than 10 mg of the orthophosphate salt of (1RS,3RS,6RS)-6-dimethylaminomethyl-1-(3-methoxyphenyl)-cyclohexane-1,3-diol are suspended for 2 days at 50° C. in acetonitrile, the supernatant solution is filtered off, the acetonitrile is slowly evaporated, and the solid thereby obtained is dried in vacuo for 1 day at room temperature.

The application furthermore provides polymorph C obtainable by the aforedescribed process.

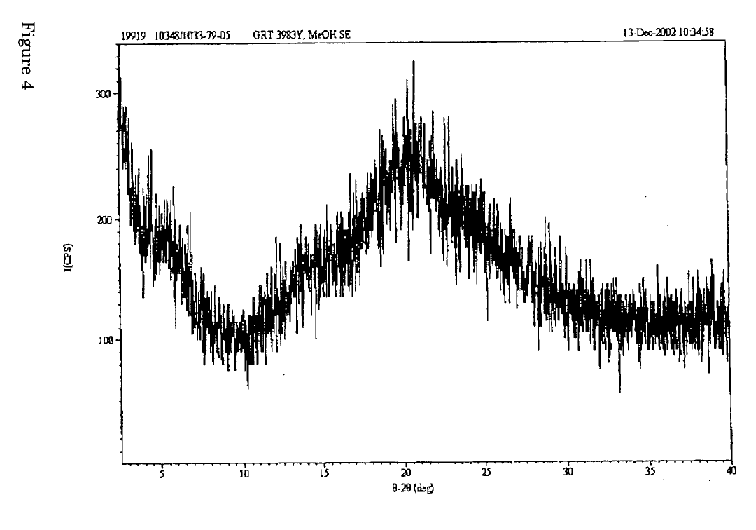

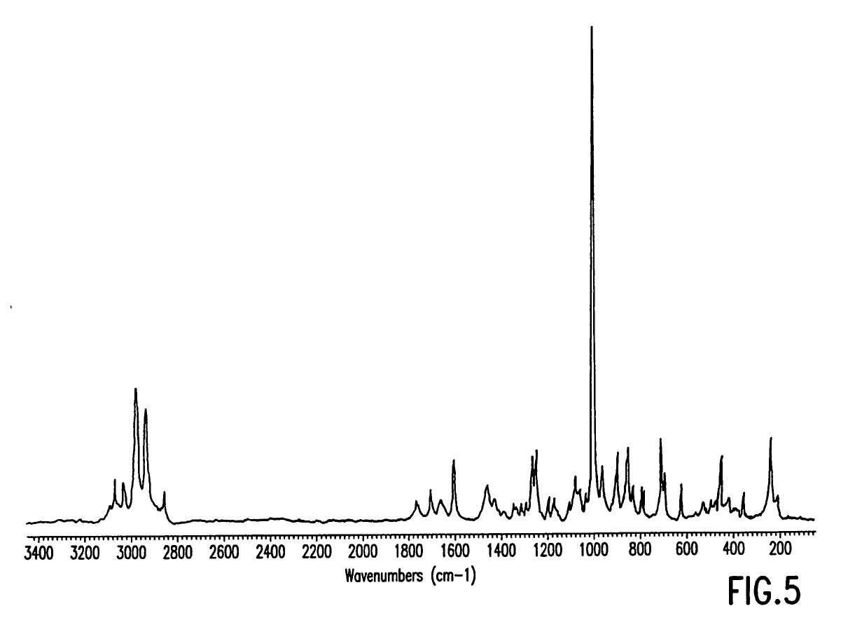

The application furthermore provides the “amorphous” polymorph of the orthophosphate I-P, characterized by a powder diagram as shown in FIG. 4, measured with Cu Kalpha radiation.

The application furthermore provides a process for the preparation of “amorphous” polymorph, according to which polymorph B is dried at a temperature of >50° C., preferably under reduced pressure.

In a preferred embodiment of the process polymorph B may be dried in vacuo for a period of ≧24 hours, preferably ≧48 hours, particularly preferably ≧72 hours, at a temperature of >60° C., preferably at ca. 68° C.

The application furthermore provides “amorphous” polymorph obtainable according to one of the aforedescribed processes.

### Synthesis Examples and Characterization of I-H and I-P

Processes and methods

RT means room temperature, m.p. melting point.

Unless described otherwise, the procedure was as follows in the experiments with slow and rapid evaporation of the solvent in the examples for synthesis of the phosphate salts according to the invention.

Approx. 30-50 mg of the phosphate salt of I are treated with approx. 100 μl of the solvent. For faster dissolving of the samples, the sample was treated in an ultrasonic bath between the addition steps.

An amount of the particular stated solvent was added until the samples, on visual inspection, were dissolved completely. Thereafter, the solution was filtered through a 0.2 μm filter, which was attached to an injection syringe.

A distinction was then made between two procedures in the subsequent course.

In order to remove the solvent rapidly, the sample was stored in a test tube at room temperature without being covered in order to achieve rapid evaporation of the solvent. In order to remove the solvent slowly, the sample, in a test tube at room temperature, was covered with a film in which some holes were made with the aid of a needle. It was thus possible for the evaporation of the solvent to take place more slowly compared with the open sample.

Unless stated otherwise, vacuum in the following is to be understood as meaning a vacuum in the range of from approx. 10 to approx. 150 mbar.

Apparatus

The powder diffractograms were recorded by means of a STOE Stadi P, Shimadzu XRD-6000 or Inel XRG-3000.

Stoe Stadi P
Diffractometer: transmission
Monochromator: curved, germanium(III)
Wavelength: Cu Kα radiation
Detector: linear PSD
Scan mode: transmission/moving PSD/fixed omega
Scan type: 2θ:omega (2θ:2°-50°, step 0.5°; omega 1°-25°, step 0.25°, time/step 30 s

Shimadzu XRD-6000
Cu Kα radiation
NaI scintillation detector. θ-2θ continuous scan with 3°/min (0.4 sec/0.02° step) from 2.5 to 40°2θ.

Inel XRG-3000 diffractometer
Detector: CPD (curved position sensitive), 2θ to 120°
Wavelength: Cu Kα radiation
Resolution: 0.03° (2θ)
Recording: 2.5-40° (2θ)

Differential scanning calorimetry

Unless stated otherwise, the DSC analyses were carried out in a TA Instruments 2920 differential scanning calorimeter or a Mettler-Toledo DSC 821 for differential thermoanalysis.

The samples were weighed into an aluminum crucible, which was closed with a perforated cover.

The samples were as a rule analyzed in the range of from 25° C. to 250° C. or 350° C. in a stream of nitrogen. The heating rate was 10° C./min.

Modulated DSC data were recorded on a TA Instruments 2920, which is equipped with a cooling system.

The samples were weighed into an aluminum crucible, which was closed with a cover, but not crimped. The modulation amplitude was ±0.8° C. and a 60 s period with an underlying heating rate of 1° C./min from 0-150° C.

Thermogravimetric analysis

The TGA analyses were carried out by means of a TA Instruments 2950 thermogravimetric analyzer or Mettler-Toledo TGA/SDTA851. Isothermal TG in a TA Instruments 2050.

The samples were weighed into an aluminum crucible and heated up under nitrogen in a temperature range of 25—approx. 200° C. or 350° C. with a heating rate of 10°C./min.

Raman spectrometry

FT-Raman spectra were recorded with an FT-Raman 960 spectrometer (Thermo Nicolet). The excitation wavelength of the laser was 1064 nm. The output of the Nd:YVO₄ laser during irradiation of the samples was approx. 0.5 W. A germanium (Ge) detector was used as the detector. For the analysis, the samples were placed in a glass tube or in a 0.8 mm glass capillary in a holder coated with gold. 128 or 256 scans were totaled, the wavelength range was 98-3600 cm⁻¹ at a spectral resolution of 4 cm⁻¹, using a Happ-Genzel apodization.

Infrared (IR) spectroscopy

Infrared spectra were recorded with a Magna-IR 860 Fourier-Transform infrared (FT-IR) spectrometer (Thermo Nicolet). The instrument comprises an Ever-Glo mid/far IR radiation source, an “extended range” potassium bromide beam splitter and a DTGS (deuterated triglycine sulfate) detector. A Thermo Spectra-Tech collector was also used. For a spectrum, 128 or 256 scans were totaled, the resolution was approx. 1-4 cm⁻¹.

The samples were mixed with dry KBr in a weight ratio of from 99:1 to 97:3 (KBr to sample). For the measurement, the sample was introduced in a sample carrier approx. 1.3 cm in size. The background spectrum was measured on a KBr sample in order to plot a log 1/R spectrum.

NMR spectroscopy

¹H-NMR spectra in solution were recorded at room temperature with a Bruker Instruments AM-250 spectrometer. Approx. 5 mg of the sample material were conventionally dissolved in approx. 0.5 ml DMSO-d6 (NMR grade), to which approx. 0.03% (v/v) tetramethylsilane was added.

### COMPARISON EXAMPLE 1

The preparation of the hydrochloride salts I-H was carried out in accordance with the instructions in EP-B 0753506 in Example 18:

### Synthesis of Samples H1 to H3

### Comparison Example Sample H2

The preparation of the base I was carried out as described in the patent EP0753506 under Example 18. 13.83 kg of a solution of base I in acetone, corresponding to 6.09 kg of pure base I, 25 l acetone and 3.18 l water were initially introduced at 25±5° C., while stirring, into a 100 l double-walled reaction unit with an electrical anchor stirrer, PT100 temperature-measuring device and oil-based cooling/heating system. The mixture was heated to 50±5° C. and stirred at this temperature for 30±15 minutes. It was then cooled to 3±2° C. and 1.81 l 37% strength hydrochloric acid were slowly metered in such that the temperature did not exceed +10° C. The product was crystallized at 5±2° C. in the course of 22 hours.

The solid which had precipitated out was then centrifuged off and first dried at 50° C. in vacuo (pressure below 150 mbar) for 21 hours and finally dried at 130° C. in vacuo (pressure below 150 mbar) for 18 hours.

Yield: 4.34 kg (63%).

Evaluation of the X-ray powder diffractogram shows the presence of forms A, C and D of the hydrochloride salt I-H.

Differential thermoanalysis shows three endotherms, peak temperatures at approx. 110° C., approx. 133° C., approx. 200° C. and 207° C.

Thermogravimetric analysis shows no decrease in weight up to decomposition.

### Comparison Example Sample H1

1.3 g of the hydrochloride salt H2 are dried in a Petri dish in a vacuum drying cabinet at 140° C. for 46 h.

Evaluation of the X-ray powder diffractogram shows the presence of forms A and D of the hydrochloride salt.

Differential thermoanalysis shows three endotherms, peak temperatures at approx. 133° C., approx. 200° C. and 206° C.

Thermogravimetric analysis shows no decrease in weight up to decomposition.

### Comparison Example Sample H3

501.3 mg H2 are weighed into a Petri dish and stored at approx. 95% (±5) relative atmospheric humidity at room temperature for 180 hours. The sample is then stored in the presence of drying beads at approx. 5% (±5) relative atmospheric humidity for a further approx. 6 hours.

According to thermogravimetric analysis, the water content of the sample is approx. 5%.

### EXAMPLES ACCORDING TO THE INVENTION

### Example 1

Sample P3

The liberation of the base from the hydrochloride salt I-H was carried out as follows:

27.69 g of the hydrochloride salt of base I are dissolved in approx. 140 ml distilled water in a 500 ml three-necked flask, the solution is cooled to approx. 15° C. and sodium hydroxide solution (32% strength) is added at a temperature below 25° C. until a pH of 11 is reached. During this procedure, the mixture is stirred continuously with a compressed air stirrer with a PTFE blade stirring rod. After addition of 10 ml of sodium hydroxide solution, a white oily solid precipitates out, which is partially dissolved by addition of approx. 10 ml ethyl acetate, before further addition of alkali, to improve the stirring. After addition of 20 ml, a pH of 11 is reached. The pH was tested by means of pH paper.

For working up, the base I is extracted with ethyl acetate, dried over magnesium sulfate and evaporated on a rotary evaporator in vacuo.

The solution evaporated to half on the rotary evaporator is left to stand at room temperature for approx. 5 days.

During this time colorless crystals up to 1 cm in size have formed on the bottom of the flask, and these are filtered off, and rinsed off with a little cold ethyl acetate (solid 1). The solution is concentrated to dryness. A beige solid (solid 2) remains in the flask.

Analysis:

Solid 1: M.p.: 134.1° C.

Solid 2: M.p.: 118.0° C.

Yield:

Solid 1: 6.07 g, 24.8% of theory

Solid 2: 16.49 g, 67.3% of theory

Total yield: 92.1% of theory.

The conversion into the phosphate salt P3 was carried out in accordance with the following instructions.

11.58 g of the crude base I are suspended in approx. 58 ml ethanol in a 250 ml three-necked flask, the suspension is cooled to about 0-10° C. and a solution of approx. 4.84 ml phosphoric acid (concn.=approx. 85 wt. % ) in approx. 29 ml distilled water is slowly added by means of a dropping funnel such that the temperature does not exceed 10° C. During this procedure, the mixture is stirred with a compressed air stirrer with a PTFE blade stirring rod. After addition of approx. 5 ml of the dilute phosphoric acid, the suspension mostly becomes clear, and after addition of approx. 8 ml, a white solid precipitates out. When the addition has ended, the reaction mixture is stirred in an ice-bath overnight. During the stirring, the ice of the ice-bath melts and the reaction temperature rises slowly to room temperature. The solid which has precipitated out is filtered off over a G3 glass filter funnel and dried in vacuo.

Yield:

9.85 g (63% of th.), white solid P3

The X-ray powder diffractogram shows form A.

### Example 2

### Phosphate Salt Sample P1

17.5 g base I were suspended in 55 ml ethanol in a 250 ml round-bottomed flask, and dilute phosphoric acid (7.33 g 89% strength phosphoric acid in 45 ml water) was added. For crystallization, the batch was seeded with phosphate salt of I and stirred at 5-7° C. for 3.5 hours. The crystals which had precipitated out were then filtered off with suction over a G3 glass frit and dried in a drying cabinet under 60-80 mbar and at a temperature in the range of from 40 to 45° C. for approx. 16 hours.

Yield: 10.92 g (46%).

For recrystallisation from ethanol, 10.9 g of the phosphate salt from Example 2 were suspended in approx. 50 ml ethanol in a 250 ml single-necked flask, and dissolved in a total of about 100 ml ethanol at the boiling point with a reflux condenser attached to the flask. The mixture was cooled to room temperature, while stirring slowly with a magnetic stirring rod. A white solid precipitated out at approx. 60° C. and the suspension was therefore heated again to the boiling point, and a further 70 ml ethanol were added. The solution was allowed to cool, while stirring slowly (a white solid precipitated out at approx. 40° C.). After reaching room temperature, the mixture was cooled down slowly in an ice-bath and the temperature was then kept at approx. 4° C.

After approx. 16 hours, the solid which had precipitated out was filtered off with suction over a glass filter funnel and dried to constant weight in a vacuum drying cabinet at approx. 60° C. and under a vacuum of approx. 70-120 mbar for approx. 2 hours.

The material was crystalline. Rod-shaped crystals up to approx. 0.2 mm long were to be seen in the sample.

Yield: 9.47 g P1 (87% of theory)

Analysis:

The content (referred to the base) is determined as 69.3% by means of HPLC.

The purity is determined as approx. 95.1% by means of HPLC.

The X-ray powder diffractogram shows the presence of form A.

### Example 3

### Phosphate Salt Sample P2

17.5 g base I were suspended in 55 ml ethanol in a 250 ml round-bottomed flask, and dilute phosphoric acid (7.33 g 89% strength phosphoric acid in 45 ml water) was added. For crystallization, the batch was seeded with phosphate salt of I and stirred at 5-7° C. for 3.5 hours. The crystals which had precipitated out were then filtered off with suction over a G3 glass frit and dried in a drying cabinet under 60-80 mbar and at a temperature in the range of from 40 to 45° C. for approx. 16 hours (see Example 2).

For further purification, 1.5 g of this phosphate salt are initially introduced into approx. 8 ml of an ethanol/water mixture (9:1 vol./vol.) in a 25 ml single-necked flask and the mixture is heated to the boiling point with a reflux condenser attached to the flask, while stirring with a magnetic stirring rod. The solvent mixture is added until a clear solution exists (total volume approx. 11.5 ml). The solution is cooled to room temperature, while stirring slowly. After approx. 5 minutes, a white solid crystallizes out. The suspension is then subsequently stirred in an ice-bath.

After 4 hours, the white solid which has precipitated out is filtered off over a G4 glass filter funnel and suctioned dry. The solid is then dried in a vacuum drying cabinet at 25° C. overnight.

Yield: 1.438 mg (95.8% of th.)

White crystalline solid.

The Raman spectrum and X-ray powder diffractogram show the presence of form A.

### Example 4

For purification, 0.5 g P1 is initially introduced into 3 ml ethanol/water (9:1 vol./vol.) in a 25 ml single-necked flask and the mixture is heated to the boiling point with an air condenser attached to the flask. The solution is cooled to room temperature, while stirring with a magnetic stirring rod. After approx. 5 minutes, a fine white solid precipitates out. 1 ml solvent is added, so that the suspension becomes stirrable. This is then subsequently stirred overnight at approx. 4° C.

After 16 hours, the white crystalline solid which has precipitated out is filtered off by means of a G4 glass filter, washed once with 2 ml of a cold ethanol/water mixture and suctioned dry. After complete drying in air, the yield is determined.

Yield: 431 mg (86.2%)

According to HPLC purity analysis, the sample comprises 100% of the phosphate salt of I.

Analysis by means of differential thermoanalysis showed an endotherm at approx. 125° C., an endotherm at approx. 139° C. and then decomposition from approx. 200° C. The X-ray powder diffractogram shows form A.

### Example 5

The robustness of the synthesis manifests itself by a possible variation in the stoichiometric ratios of base to acid. In this example, the ratio of base:acid=approx. 1:1. 1.4 g base I are initially introduced into 7 ml ethanol in a 25 ml two-necked flask. The pH at the start of the experiment was approx. pH=9.2. 2.4 ml of a phosphoric acid solution (concn.=2 mole/l) were added in 200 μl steps to the solution initially introduced, while stirring with a magnetic stirring rod, and the pH was measured.

After addition of 600 μl of the phosphoric acid solution, the suspension became clear. After 1.4 ml (pH: 7.3), the solution was stirred for approx. 40 minutes without further addition. A white solid has precipitated out and the suspension has a pH of approx. 8.7. The acid is added again in 200 μl steps. When the addition has ended, the mixture is subsequently stirred for approx. 1 hour and the solid is then filtered off over a tared G4 glass filter, washed once with approx. 4 ml ethanol and dried by means of a vacuum being applied. After approx. 30 minutes, the solid is introduced into a test tube.

Yield: 1.886 g (99.7% of th.), white solid

In the differential thermoanalysis, an endotherm manifested itself at approx. 134° C. The X-ray powder diffractogram shows form A. The weight loss was determined at 4.21% in the range of 30-150° C. by means of thermogravimetry.

TABLE
Course of the titration with phosphoric acid

| Volume of phosphoric acid | pH |
|---|---|
| 0.0 | 9.22 |
| 0.2 | 8.70 |
| 0.4 | 8.06 |
| 0.6 | 7.67 |
| 0.8 | 7.38 |
| 1.0 | 7.47 |
| 1.2 | 7.43 |
| 1.4 | 7.3 |
| 1.6 | 8.68 |
| 1.8 | 8.55 |
| 2.0 | 8.4 |
| 2.2 | 8.04 |
| 2.4 | 5.8 |

### Example 6

The robustness of the synthesis manifests itself by a possible variation in the stoichiometric ratios of base to acid. In this example, the ratio of base:acid=approx. 1:2. 1.4 g base I are initially introduced into 7 ml ethanol in a 25 ml two-necked flask. The pH at the start of the titration was pH=approx. 9.1. 5 ml of a phosphoric acid solution (concn.=2 mole/l) were added in 200 μl steps and the pH and the temperature were measured.

After addition of 2.4 ml (pH=7.0), the solution was stirred for approx. 40 minutes without further addition. A white solid has precipitated out and the suspension had a pH of approx. 8.7 after this time. The addition of the acid in 200 μl steps was continued. When the addition had ended, the mixture was subsequently stirred for 1 hour and the solid was then filtered off through a G4 glass filter, washed once with approx. 4 ml ethanol and suctioned dry by means of a vacuum being applied. The filtrate was discarded. After approx. 30 minutes, the solid is introduced into a test tube.

Yield: 0.735 g (38.9% of th.), white solid

In the differential thermoanalysis, an endotherm manifested itself at approx. 134° C. The X-ray powder diffractogram shows form A. The weight loss was determined at 4.12% in the range of 30-170° C. by means of thermogravimetry.

TABLE
Course of the titration with phosphoric acid

| Volume of phosphoric acid | pH | Temperature |
|---|---|---|
| 0.0 | 9.09 | 21.3 |
| 0.2 | 9.15 | |
| 0.4 | 9.32 | |
| 0.6 | 9.35 | |
| 0.8 | 9.29 | 24.3 |
| 1.0 | 9.18 | 24.6 |
| 1.2 | 9.08 | 25.2 |
| 1.4 | 8.93 | 25.7 |
| 1.6 | 8.74 | 26.3 |
| 1.8 | 8.56 | 26.8 |
| 2.0 | 8.31 | 27.2 |
| 2.2 | 7.98 | 27.5 |
| 2.4 | 7.01 | 27.7 |
| 2.6 | 4.54 | 27.7 |
| 2.8 | 4.23 | 27.4 |
| 3.0 | 4.04 | 26.8 |
| 3.2 | 3.90 | 26.5 |
| 3.4 | 3.80 | 26.3 |
| 3.6 | 3.50 | 26.2 |
| 3.8 | 3.38 | 25.9 |
| 4.0 | 3.35 | 25.7 |
| 4.2 | 3.31 | 25.6 |
| 4.4 | 3.28 | 25.4 |
| 4.6 | 3.25 | 25.3 |
| 4.8 | 3.21 | 25.1 |
| 5.0 | 3.20 | 25 |

### Example 7

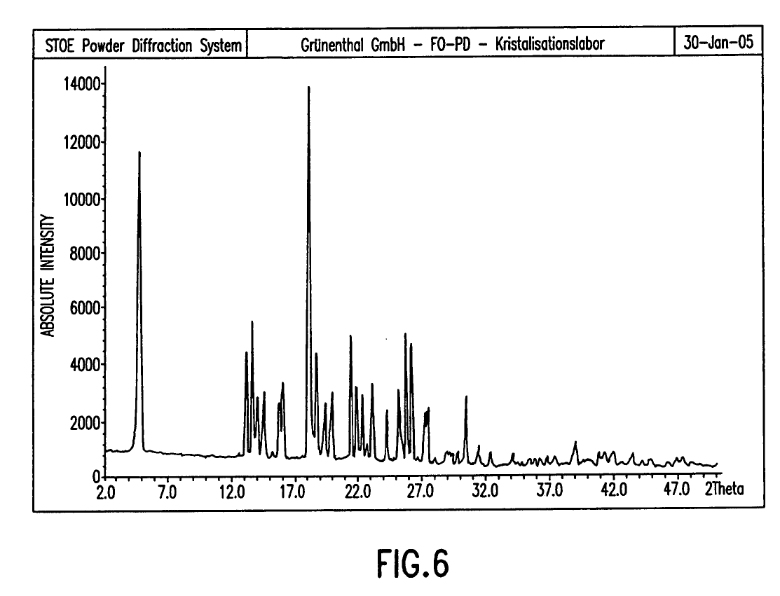

X-ray powder diffractogram of the phosphate salt of I P3
cf. FIG. 6
Peak list in Table 5
Raman spectrum of the phosphate salt of I P3
Spectrum with base line correction
cf. FIG. 5

### Example 8

The robustness of the synthesis manifests itself by a possible variation in the stoichiometric ratios of base to acid and the use of various solvents.

In this example, the ratio of base:acid=approx. 2:1.

14.5 ml ethyl ether were added to 151,4 mg base I. The suspension was treated in an ultrasonic bath in order to dissolve the solids. The solution was then stirred with a magnetic stirring rod. Phosphoric acid (30.6 mg, concn.=85.7%) was diluted with 2 ml ethyl ether and added dropwise to the solution of base I in ether. The suspension was stirred for 30 min and the solid was subsequently isolated by means of filtration and then dried in vacuo. The yield was 72.1 mg.

The precipitate was identified as form A by means of X-ray powder diffractometry.

### Example 9

The robustness of the synthesis manifests itself by a possible variation in the stoichiometric ratios of base to acid and the use of various solvents.

In this example, the ratio of base:acid=approx. 2:1.

603.9 mg base I were weighed into a 50 ml round-bottomed flask. 50 ml ethyl ether were added to this and the suspension was then stirred with a magnetic stirring rod. Phosphoric acid (123.6 mg, concn.=85%) was diluted with 0.5 ml methanol and added dropwise to the solution of base I in ether. The remainder of the phosphoric acid solution was rinsed into the round-bottomed flask by addition of a little ethyl ether. On addition of the phosphoric acid solution to the solution of the base, the formation of a solid was observed. The suspension was stirred for 15 min and isolated by filtration. Drying (3 h, vacuum centrifuge) resulted in 322.7 mg of a solid. X-ray powder diffractometry showed form A. Thermogravimetry showed a weight loss of 5.2 wt. %.

### Example 10

For formation of the polymorph form B, approx. 30-50 mg of the phosphate salt of I were treated with approx. 100 μl of the solvent. For faster dissolving of the samples, the sample was treated in an ultrasonic bath between the addition steps. An amount of solvent was added until the samples, on visual inspection, were dissolved completely. The solution was then filtered through a 0.2 μm filter attached to an injection syringe and stored in a test tube at room temperature, without being covered, in order to achieve rapid evaporation of the solvent.

The solid formed was collected after complete evaporation of the solvent.

To dry the sample completely, the latter can optionally additionally be dried at room temperature in vacuo.

The synthesis was carried out in a mixture of acetonitrile/ethanol in the ratio of the volumes of 75:25 (vol-vol. % ). Since the diffractogram of the sample obtained from the rapid evaporation showed a so-called preferred orientation (abbrevn.: PO) (cf. FIG. 1), the sample was ground carefully in order to obtain a representative diffractogram of the material (cf. FIG. 2).

The X-ray powder diffractogram shows form B. Differential thermoanalysis shows an endotherm at approx. 117° C., an endotherm at approx. 145° C. and an endotherm at approx. 150° C.

Weight loss according to thermogravimetric analysis approx. 8.5% up to approx. 160° C.

Karl Fischer titration showed a water content of approx. 5.9 wt. % .

¹H-NMR spectroscopy showed a content of approx. 0.2 mole acetonitrile per molecule of base.

X-ray powder diffractogram form B

cf. FIG. 2 in the appendix

Form B of the phosphate salt of I differs from form A on the basis of the thermal data, X-ray powder diffractogram, ¹H-NMR spectrum and Raman spectrum.

Thermal analysis of form B shows two relatively large endotherms at approx. 117° C. and approx. 145° C., and a smaller endotherm at approx. 150° C.

Thermogravimetry showed a weight loss of 7.75% up to approx. 132° C. A water content of 5.9 wt. % was found for form B by means of Karl Fischer titration. The acetonitrile content was then concluded from the difference between these two values.

Both the infrared and the Raman spectrum of form A differ from the spectra of form B.

The Raman spectrum of form B shows the presence of acetonitrile by a peak at approx. 2249 cm⁻¹.

The infrared spectrum of form B shows the presence of acetonitrile by a peak at approx. ˜2247 cm⁻¹

The infrared spectrum of form B shows some other peaks which do not occur with form A.

The different amount of acetonitrile found after drying experiments by means of evaluation of the NMR spectra are an indication that form B is possibly a variable solvate.

Form B was obtained exclusively from samples which had been prepared only with the solvent acetonitrile or in which this had the larger content in the solvent mixture.

### Example 11

Form B could be reproducibly prepared by suspending form A for 6 days at room temperature in acetonitrile.

### Example 12

Form B could be reproducibly prepared by suspending form A for 2 days at 50° C. in acetonitrile.

### Example 13

The form B was dried for 13 hours at 40° C. in vacuo. Based on X-ray powder diffractometry, thermal analysis and ¹H NMR spectroscopy no changes in the physical form were observed.

The acetonitrile content was, after the treatment, ca. 2.5%, as was also shown from an evaluation of the ¹H NMR spectrum.

### Example 14

The form B was dried for 24 hours at 40° C. in vacuo. Based on X-ray powder diffractometry, thermal analysis and ¹H NMR spectroscopy no changes in the physical form were observed.

The acetonitrile content was, after the treatment, ca. 2.5%, as was also shown from an evaluation of the ¹H NMR spectrum.

### Example 15

Form B was dried at 68° C. in a drying cabinet in vacuo for 3 days. The sample converted into amorphous material.

After this treatment, no acetonitrile remained in the sample.

### Example 16

A few milligrams (less than 10 mg) of the phosphate salt of I are suspended in acetonitrile at 50° C. for 2 days.

The supernatant solution is filtered off and the solvent is evaporated slowly.

The solid obtained in this way was subjected to vacuum drying at room temperature for one day before the analysis.

From the comparison of the X-ray diffractogram with that of forms A and B, it is found that some peak positions indeed coincide, but further peaks occur, which indicate that it is rather a new form, called form C, than a mixture of form A and form B.

The X-ray powder diffractogram of form C is shown in FIG. 3 in the appendix.

### Example 17

10 mg of the phosphate salt of I are suspended in acetonitrile at 50° C. for 2 days.

The supernatant solution is filtered off and the solvent is evaporated slowly.

The solid sample obtained was dried at room temperature in vacuo for 1 day.

The X-ray diffractogram of the solid obtained in this way corresponded to that of form A. The Raman spectrum showed that the sample also contained acetonitrile.

### Example 18

It was possible to prepare the amorphous form of the phosphate salt of I by drying form B at 68° C. in vacuo for approx. 3 days.

### Example 19

The amorphous phosphate salt of I crystallized during a DVS (dynamic vapor sorption) experiment at a relative atmospheric humidity of 45% r.h. The hydrate formed in this way (form A) remained stable during the desorption.

### Example 20

The amorphous phosphate salt crystallized after 8 days on storage at a relative atmospheric humidity of 75% r.h. and formed form A.

Differential thermoanalysis shows an endotherm at approx. 57° C., the weight loss in the thermogravimetry is approx. 1.82% up to 100° C.

### Example 32

Microscopic examination of the amorphous phosphate salt in a heating chamber shows that it starts to melt at approx. 105.6° C., and is melted completely at approx. 106.2° C. During the heating and cooling for the microscopic analysis in the heating chamber, no recrystallisation at all occurred.

The glass transition temperature was found to be approx. 86.7° C. by means of modulated differential thermoanalysis.

### Example 21

Summarizing overview of the diffractograms of the polymorphic forms

The crystalline forms and the amorphous form can be differentiated with the aid of the powder diffractograms.
cf. FIGS. 1 to 4 in the appendix.
cf. Table 1 to 3 in the appendix.

### Example 22

Comparative comparison of the infrared spectra of the polymorphic forms A and B.

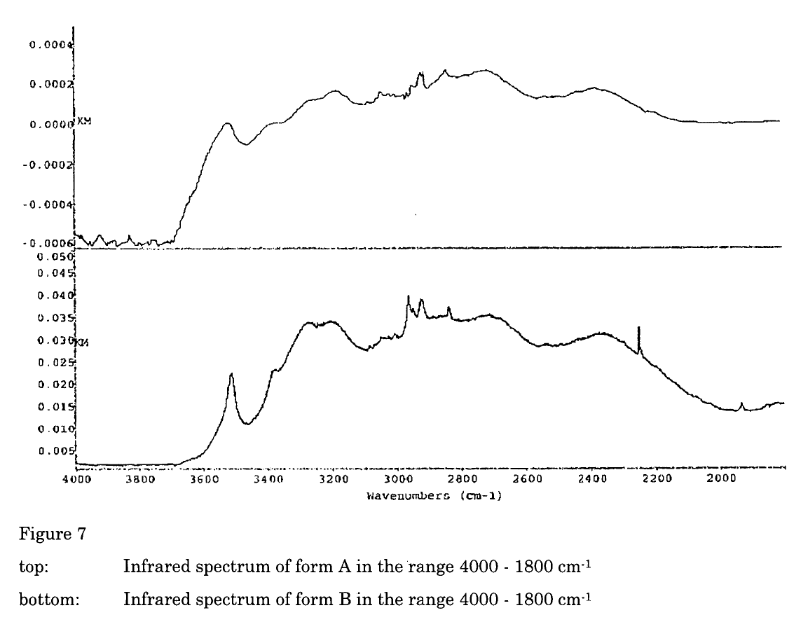

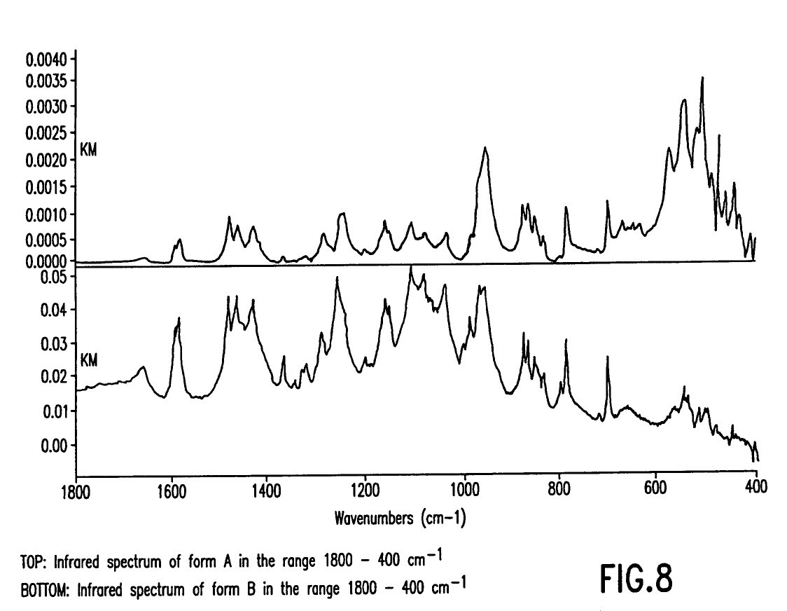

Forms A and B can be differentiated with the aid of the infrared spectra.
cf. FIGS. 7 and 8 in the appendix.

### Example 23

Comparative comparison of the Raman spectra of the polymorphic forms A and B

Forms A and B can be differentiated with the aid of the Raman spectra.

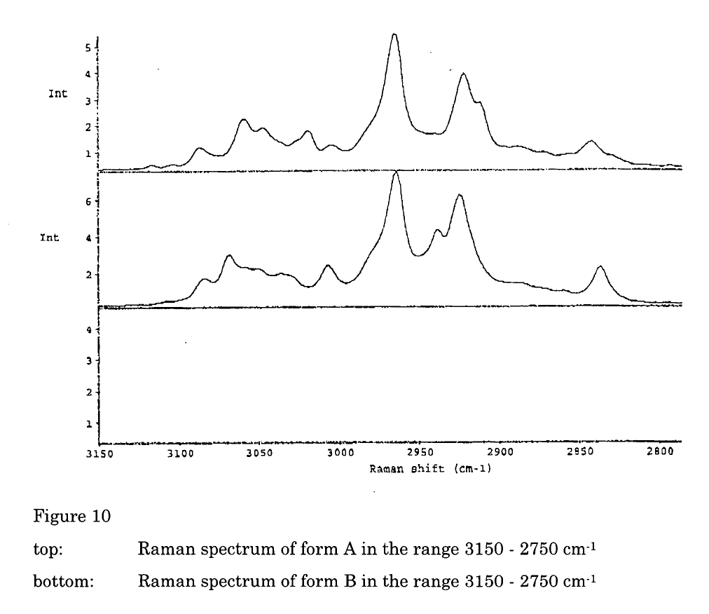

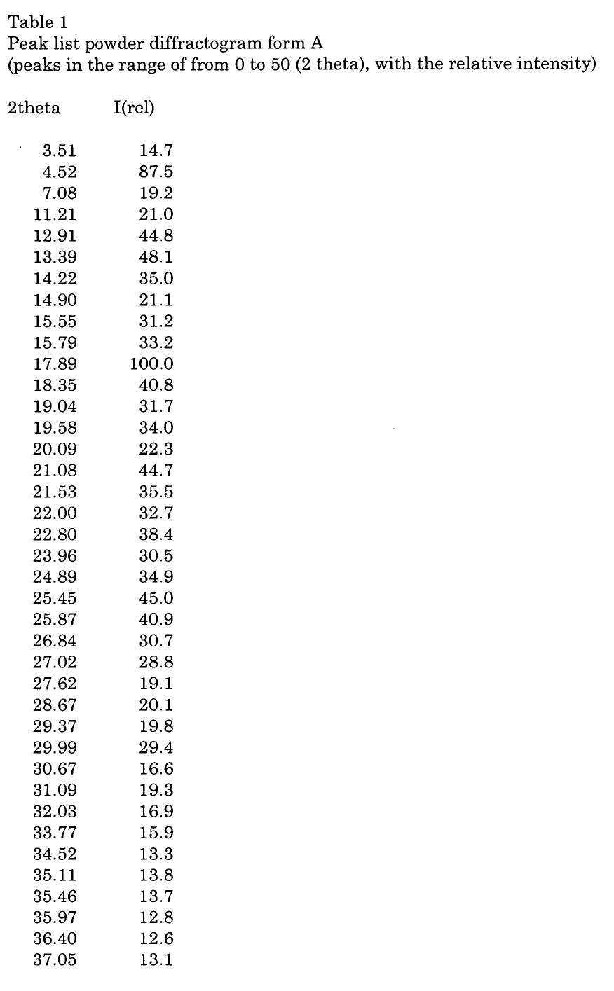

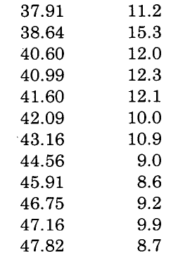

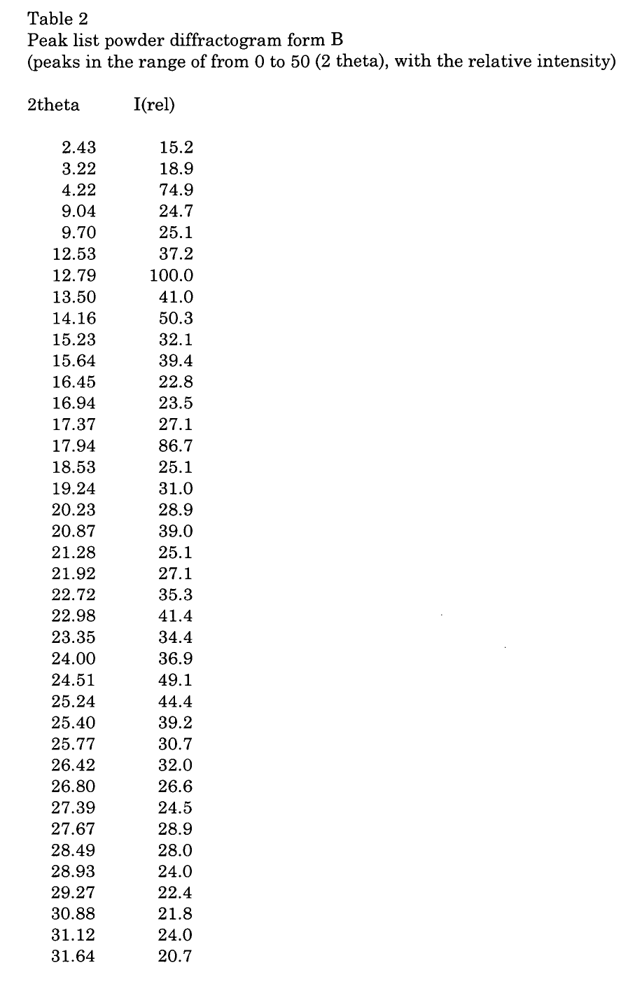

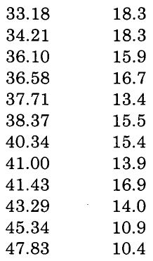

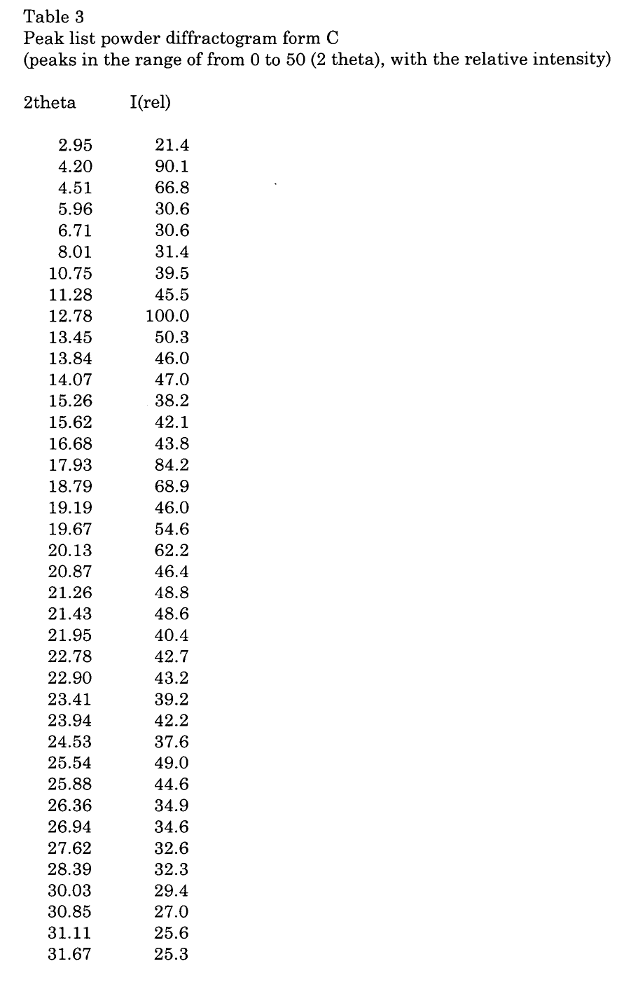

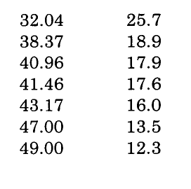

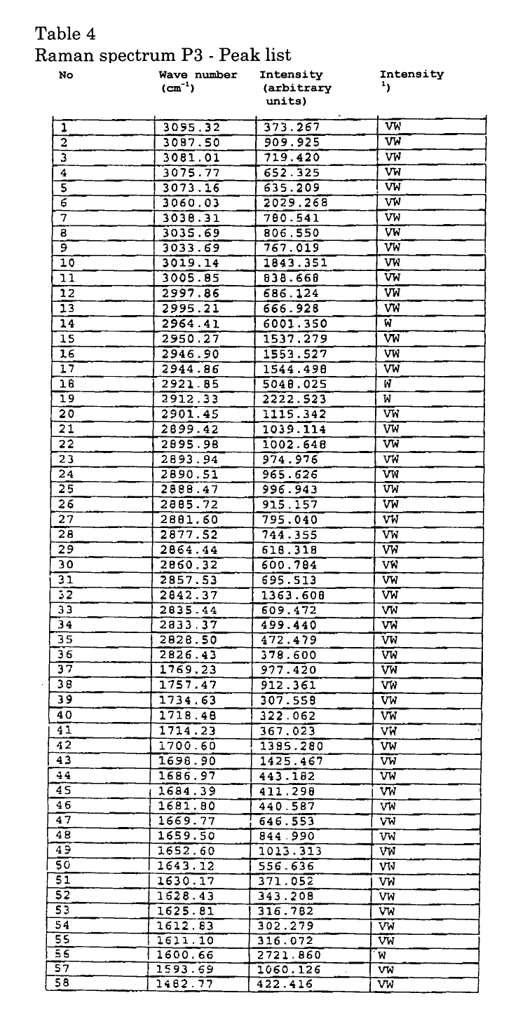

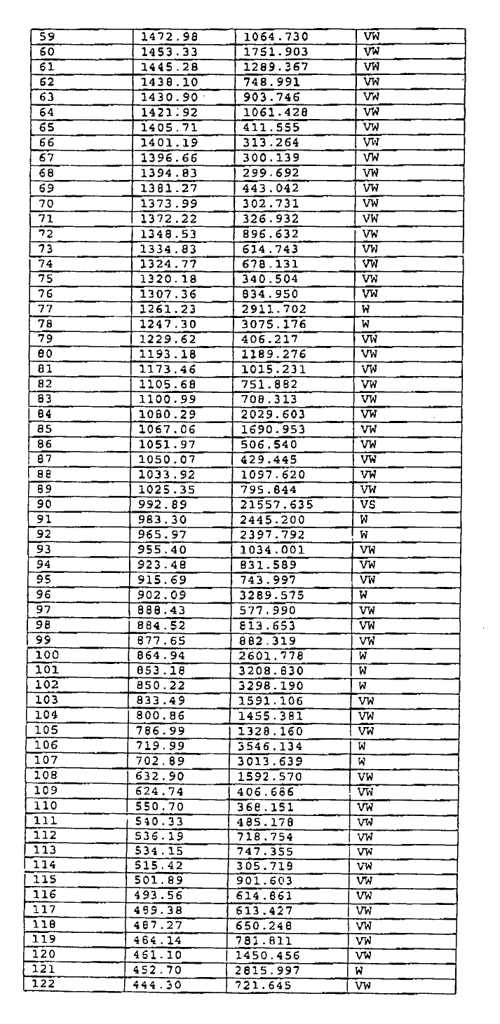

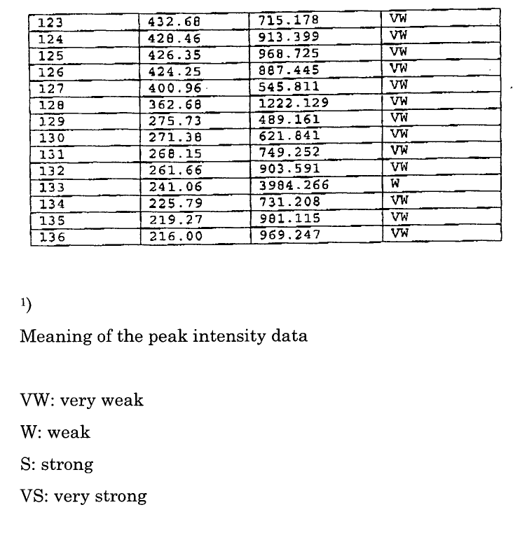

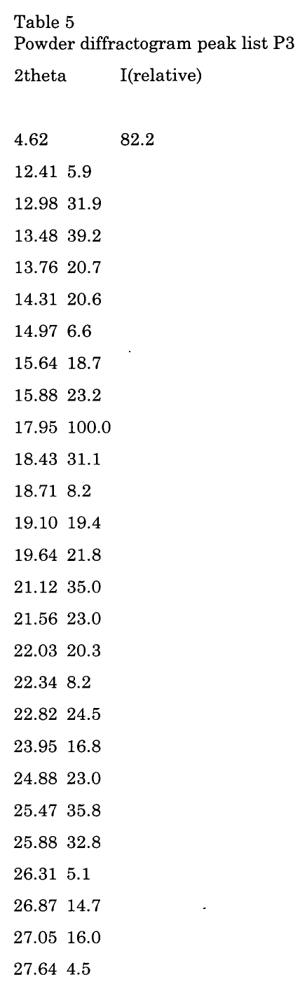

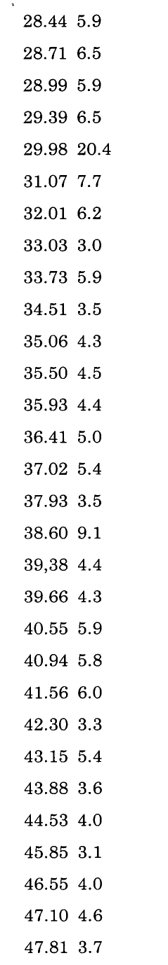

cf. FIGS. 9 and 10 in the appendix.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereto.

What is claimed is:

1. A 6-dimethylaminomethyl-1-(3-methoxyphenyl)-1,3-dihydroxy-cyclohexane compound corresponding to formula (I) 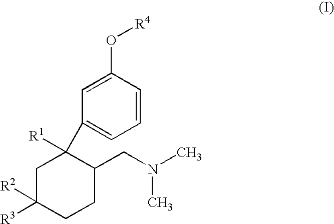
wherein
R¹ denotes OH and
R² denotes OH and R³ denotes H or
R³ denotes OH and R² denotes H and
R⁴ denotes CH₃
in the form of a salt of phosphoric acid.

2. The compound of claim 1, wherein said compound is in the form of a salt of a diphosphoric acid or an orthophosphoric acid or a combination thereof.

3. The compound of claim 1, wherein the phosphoric acid is orthophosphoric acid.

4. The compound of claim 1, wherein the compound has a configuration corresponding to formula Ia

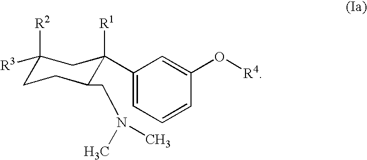

5. The compound of claim 1, wherein R¹ and R² in each case denote OH, R³ denotes hydrogen and R⁴ denotes CH₃.

6. The compound of claim 5, wherein said compound is present in the form of a racemic mixture.

7. The compound of claim 6, wherein said compound is (1RS,3RS,6RS)-6-dimethylaminomethyl-1-(3-methoxyphenyl)-cyclohexane-1,3-diol.

8. The compound of claim 1, wherein said compound is (+)-(1R,3R,6R)-6-dimethylaminomethyl-1-(3-methoxyphenyl)-cyclohexane-1,3-diol or (−)-(1S,3S,6S)-6-dimethylaminomethyl-1-(3-methoxyphenyl)-cyclohexane-1,3-diol.

9. A process for preparing the compound of claim 1, comprising the step of reacting a compound corresponding to formula (I) with phosphoric acid in a reaction medium.

10. The process of claim 9, wherein said compound corresponding to formula (I) is provided in the form of a hydrochloride or a free base.

11. The process of claim 10, wherein said compound corresponding to formula (I) is provided in a molar ratio of compound to phosphoric acid of from 2:1 to 1:2.

12. The process of claim 10, comprising:
providing said compound corresponding to formula (I) in the form of a free base,
suspending said compound at 10-40° C. in alcohol,
adding dilute phosphoric acid and
stirring the mixture at 0-10° C.

13. The process of claim 12, wherein said alcohol is isopropanol or ethanol.

14. The process of claim 12, further comprising the step of seeding the mixture with a phosphate salt of the compound corresponding to formula (I) at 0-10° C.

15. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram containing one or both of the following reflections: 30.0 and 33.7 (in each case ±0.2 2θ).

16. The polymorph of claim 15, said polymoroph also exhibiting one or more of the following reflections: 4.6, 13.8, 15.6, 15.9, 18.0, 18.4, 19.1, 19.6, 21.6, 24.9 and 32.0 (in each case ±0.2 2θ).

17. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram as shown in FIG. 1, measured with Cu Kα radiation.

18. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a Raman spectrum containing one or more of the following signals: 2912, 3020 and 3060 (in each case in cm⁻¹±4 cm⁻¹).

19. The polymorph of claim 18, also exhibiting one or more of the following signals: 2843, 2922, 2966 and 3089 (in each case in cm⁻¹±4 cm⁻¹).

20. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a Raman spectrum with an excitation wavelength at least at 1064 nm.

21. The process of claim 9 wherein said compound corresponding to formula (I) is (1RS,3RS,6RS)-6-dimethylaminomethyl-1-(3-methoxyphenyl)-cyclohexane-1,3-diol and said phosphoric acid is orthophosphoric acid and said process includes the step of isolating the resulting polymorph.

22. The process of claim 21, wherein (1RS,3RS,6RS)-6-dimethylaminomethyl-1-(3-methoxyphenyl)-cyclohexane-1,3-diol and orthophosphoric acid are provided in a molar ratio of 2:1 to 1:2.

23. The process of claim 21, wherein the reaction is carried out at a temperature of 10-40° C.

24. The process of claim 21, wherein said reaction medium comprises an alcohol.

25. The process of claim 24, wherein said reaction medium further comprises water.

26. The process of claim 24, wherein said alcohol is isopropanol or ethanol.

27. The process of claim 21, wherein the (1RS,3RS,6RS)-6-dimethylaminomethyl-1-(3-methoxyphenyl)-cyclohexane-1,3-diol and orthophosphoric acid are stirred at 0-10° C.

28. The process of claim 21, wherein the (1RS,3RS,6RS)-6-dimethylaminomethyl-1-(3-methoxyphenyl)-cyclohexane-1,3-diol and orthophosphoric acid are seeded with the polymorph at 0-10° C.

29. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol exhibiting a powder diffractogram containing one or more of the following reflections: 17.0, 17.4 and 20.2 (in each case ±0.2 2θ).

30. The polymorph of claim 29, said polymorph also exhibiting one or more of the following reflections: 4.3, 14.6, 15.2, 15.6, 18.0 and 31.6 (in each case ±0.2 2θ).

31. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram as shown in FIG. 2, measured with Cu Kα radiation.

32. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a Raman spectrum containing one or both of the following signals: 2940 and 3070 (in each case in cm⁻¹±4 cm⁻¹).

33. The polymorph of claim 32, also exhibiting one or more of the following signals: 2839, 2926, 2964 and 3084 (in each case in cm⁻¹±4 cm⁻¹).

34. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a Raman spectrum with an excitation wavelength of 1064 nm.

35. The process of claim 21 further comprising the steps of stirring the resulting polymorph in acetonitrile or in a medium comprising acetonitrile and then isolating a resulting second polymorph.

36. The process of claim 35 wherein said step of stirring the resulting polymorph in acetonitrile or in a medium based on acetonitrile is performed at elevated temperature.

37. The process of claim 35, wherein the medium contains >50 vol. % acetonitrile.

38. The process of claim 35, wherein the medium comprises an alcohol.

39. The process of claim 35, wherein the medium comprises ethanol.

40. The process of claim 35, wherein said step of stirring the resulting polymorph in acetonitrile or in a medium based on acetonitrile to form the resulting second polymorph is performed at elevated temperature.

41. The process of claim 35, further comprising the step of drying the resulting second polymorph under reduced pressure at a temperature of ≦60° C.

42. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram containing one or both of the following reflections: 10.7 and 11.4 (in each case ±0.2 2θ).

43. The polymorph of claim 42, also exhibiting one or more of the following reflections: 16.7 and 18.8 (in each case ±0.2 2θ).

44. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram as shown in FIG. 3, measured with Cu Kα radiation.

45. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram containing one or more measured peaks recited in Table 3, measured with Cu Kα radiation.

46. A process for preparing a polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram containing one or both of the following reflections: 10.7 and 11.4 (in each case ±0.2 2θ) comprising:
suspending less than 10 mg of the orthophosphate salt of (1RS,3RS,6RS)-6-dimethylaminomethyl-1-(3-methoxyphenyl)-cyclohexane-1,3-diol for 2 days at 50° C. in acetonitrile,
removing supernatant solution,
slowly evaporating acetonitrile, and
drying the resulting polymorph under vacuum for 1 day at room temperature.

47. A polymorph comprising the salt of claim 1, wherein said salt is the orthophosphate salt of (1RS,3RS,6RS)-6-dimethylaminomethyl-1-(3-methoxyphenyl)-cyclohexane-1,3-diol, exhibiting a powder diffractogram as shown in FIG. 4, measured with Cu Kα radiation.

48. The process of claim 35 further comprising the step of drying the resulting second polymorph at a temperature of >50° C.

49. The process of claim 48, wherein said drying step is performed under reduced pressure.

50. The process of claim 48, comprising drying the resulting second polymorph under vacuum for a period of ≧24 hours, at a temperature of >60° C.

51. A pharmaceutical formulation comprising at least one salt as set forth in claim 1 and one or more physiologically acceptable auxiliary substances.

52. The pharmaceutical formulation of claim 51, wherein the pharmaceutical formulation comprises one or more polymorphs selected from the group consisting of:
a polymorph of the orthophosphate salt of (1RS,3RS,6RS)-6-dimethylaminomethyl-1-(3-methoxyphenyl)-cyclohexane-1,3-diol, exhibiting a powder diffractogram containing one or both of the following reflections: 30.0 and 33.7 (in each case ±0.2 2θ);
a polymorph of the orthophosphate salt of (1RS,3RS,6RS)-6-dimethylaminomethyl-1-(3-methoxyphenyl)-cyclohexane-1,3-diol exhibiting a powder diffractogram containing one or more of the following reflections: 17.0, 17.4 and 20.2 (in each case ±0.2 2θ);
a polymorph of the orthophosphate salt of (1RS,3RS,6RS)-6-dimethylaminomethyl-1-(3-methoxyphenyl)-cyclohexane-1,3-diol, exhibiting a powder diffractogram containing one or both of the following reflections: 10.7 and 11.4 (in each case ±0.2 2θ); and
A polymorph of the orthophosphate salt orthophosphate salt of (1RS,3RS,6RS)-6-dimethylaminomethyl-1-(3-methoxyphenyl)-cyclohexane-1,3-diol, exhibiting a powder diffractogram as shown in FIG. 4, measured with Cu K′ radiation in an amount pharmaceutically effective for treating or inhibiting a condition from the group consisting of pain; migraine; depression; neurodegenerative diseases, preferably chosen from the group consisting of multiple sclerosis, Alzheimer's disease, Parkinson's disease; Huntington's disease; cognitive diseases; anxiety states; panic attacks; epilepsy; coughing; urinary incontinence; diarrhea; pruritus; schizophrenia; cerebral ischemia; muscle spasms; spasms; food intake disorders; alcohol dependency; substance dependency; drug dependency; alcohol abuse; substance abuse; drug abuse; withdrawal symptoms with alcohol, substance or drug dependency; development of tolerance to substances; and gastro-esophageal reflux syndrome; or for diuresis; for antinatriuresis; for influencing the cardiovascular system; for increasing vigilance; for increasing libido; for modulation of motor activity or for local anesthesia.

53. A method of treating or inhibiting a condition selected from the group consisting of pain; migraine; depression; neurodegenerative diseases, preferably chosen from the group consisting of multiple sclerosis, Alzheimer's disease, Parkinson's disease and Huntington's disease; cognitive diseases; anxiety states; panic attacks; epilepsy; coughing; urinary incontinence; diarrhea; pruritus; schizophrenia; cerebral ischaemias; muscle spasms; spasms; food intake disorders; alcohol dependency; substance dependency; drug dependency; alcohol abuse; substance abuse; drug abuse; withdrawal symptoms with alcohol, substance or drug dependency; development of tolerance to substances; and gastro-esophageal reflux syndrome; or for diuresis; for antinatriuresis; for influencing the cardiovascular system; for increasing vigilance; for increasing libido; for modulation of motor activity or for local anesthesia, said method comprising administering a pharmaceutically effective amount of a salt according to claim 1.
